(19) United States
(12) Patent Application Publication
(10) Pub. No.: US 2009/0318674 A1
(43) Pub. Date: Dec. 24, 2009

(54) PROCESS FOR PURIFICATION OF ANTIBODIES

(76) Inventor: Peter S. Gagnon, San Clemente, CA (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 12/477,031

(22) Filed: Jun. 2, 2009

Related U.S. Application Data
(60) Provisional application No. 61/058,545, filed on Jun. 3, 2008.

(51) Int. Cl. C07K 1/18

(52) U.S. Cl. 530/416

(57) ABSTRACT

The disclosed embodiments are directed to methods and compositions for purification of proteins, in particular, to methods and compositions for an antibody purification process that includes aggregate removal and the use of solubility enhancing additives such as zwitterion-containing compositions to enhance antibody solubility and avoid aggregate formation or occlusion during ion exchange chromatography, yielding a high-purity protein product substantially free of aggregates.

### CROSS-REFERENCE TO RELATED APPLICATIONS

The present Application claims priority from U.S. Provisional Patent Application No. 61/058,545 filed Jun. 3, 2008, which is incorporated by reference herein in its entirety.

### BACKGROUND OF THE INVENTION

1. Field of the Invention

The present disclosure relates to methods and compositions for purification of proteins, in particular, to methods and compositions for an antibody purification process that includes aggregate removal and the use of solubility enhancing additives such as zwitterion-containing compositions to enhance antibody solubility and avoid aggregate formation or occlusion during ion exchange chromatography, yielding a high-purity protein product substantially free of aggregates.

2. Introduction

IgM antibodies, found in blood and lymph fluid, are usually the first type of antibody made in response to an infection, and can cause other immune system cells to destroy foreign substances. Although IgMs have promising therapeutic applications, IgMs have some characteristics that can limit the application of standard antibody purification tools. IgMs tend to be less soluble than IgGs and more susceptible to denaturation (precipitation, including aggregate formation) at extremes of pH, and under conditions of low conductivity. IgMs arc generally tolerant of high salt concentrations, which can be useful for ion exchange chromatography, but are susceptible to denaturation from exposure to strongly hydrophobic surfaces, which can limit the usefulness of hydrophobic interaction chromatography (HIC). Furthermore, although IgMs can be eluted from moderately hydrophobic supports for HIC in a well defined peak at reasonably low salt concentration, IgMs will precipitate at the higher salt concentrations that arc preferred to support good capacity on moderately hydrophobic media. Because IgMs are typically more charged than IgGs, IgMs bind more strongly than IgGs to ion exchangers and hydroxyapatite and often bind much more strongly than most contaminants. The large size of IgMs can be a challenge for purification, due to slow diffusion constants, which can be a problem for porous particle-based chromatography media dependent on diffusion for mass transport. Slow diffusion rates can be a particular limitation for size exclusion chromatography (SEC), which already suffers from limitations of low capacity and low flow rate.

Although some characteristics of IgMs may limit the application of standard purification tools, the charge characteristics of IgM monoclonals also provide purification opportunities that arc rarely or never encountered with IgGs. These charge characteristics permit the development of orthogonal processes for purification in only a few steps, without exposing the product to unnecessary stress. In fact, purification of clinical-grade IgM can generally be achieved with three bind-elute chromatography steps on hydroxyapatite, anion exchange, and cation exchange. Much of the improvement in IgM purification comes from the use of monolithic ion exchangers with high binding capacity and the ability to tolerate rapid glow rates. Furthermore, monolith and membrane ion exchangers rely on convection for mass transport, not diffusion, and because convection is independent of size and flow rate, capacity and resolution are not affected by the large size of IgMs. Omitting an affinity step is also a positive contribution to developing purification efficient and economical purification processes. Avoiding intermediate diafiltration by using in-line dilution to load samples, can also improve process economy. At each step, recoveries are comparable to those achieved with IgG purification. (Gagnon et al., Purification of IgM Monoclonal antibodies, BioPharm International Supplements, March 2008, pages 26-35 (Mar. 2, 2008); Gagnon et al., IgM Purification: The Next Generation, 13th Annual Waterside Conference, Miami, Feb. 4-6, 2008, available at www.validated.com as Document No. PSG-080129)

Aggregate Removal

Many proteins, including antibodies such as IgGs and IgMs, can form aggregrates that must be removed during purification, in order to provide a protein product having the required purity and, for therapeutic proteins, product safety. Although aggregate removal is a key determinant of product safety, it may increase the difficulty of process development, increase purification costs, and limit the selection amongst options for final (“polishing”) purification. For example, size exclusion chromatography (SEC) removes aggregates and permits buffer exchange, but SEC is also slow, provides poor capacity, requires disproportionately large columns that require superior packing skills, and requires large buffer volumes. Adsorptive methods have limitations on their usefulness to remove aggregates, as their selectivity is not directly related to protein size, aggregates tend to be retained more strongly than non-aggregated proteins (presumably by participating in a larger number of interactions with the adsorptive solid phase), and the unpredictable degree of separation due to variations in charge distribution between clones, and between aggregated and nonaggregated forms of the product.

Nonionic polymers and proteins, often used as antibody precipitating agents, can be added to buffers to provide an effect that is proportional to protein size. Nonionic polymers and proteins can be selected to provide additives that arc compatible with adsorptive methods, enhance the ability of adsorptive methods to separate aggregates from non-aggregated antibody, and meet regulatory requirements for processing human-injectable products. For example, the nonionic polymer polyethylene glycol (PEG) is considered nontoxic, is readily available in USP grade, has protein-stabilizing properties, and is not expensive. Because PEG is preferentially excluded from protein surfaces, a pure water hydration sheath is created around the protein, and the discontinuity between the pure water sheath and the PEG-concentrated bulk solvent is thermodynamically unfavorable. When proteins come into contact in a solution of PEG, they share some hydration water with each other, thereby releasing some back to the bulk solvent, and they also present a smaller surface than the combined surface area of the individual proteins. Because protein surface area is proportional to protein size, the magnitude of the effect of nonionic organic polymers is proportional to protein size, resulting in size selectivity that can be enhanced by selection of polymer length and concentration. For example, the percentage range of PEG-6000 (as a buffer additive) that precipitates IgM is lower than the percentage range that precipitates IgG.

The size selectivity imposed by PEG can carry over to other applications, with the result that the effect of PEG can be exploited during various chromatographic separations. When PEG is included as a buffer additive under ion exchange conditions, smaller nonaggregated proteins can be separated from the larger aggregates by ion exchange. Aggregate separation can also be carried out on hydroxyapatite using PEG-containing buffers, thus allowing aggregate removal by hydroxyapatite chromatography. Because PEG effects on other contaminants usually can be predicted, these effects can be taken into account to achieve optimal clearance during product purification. For example, because host cell proteins (HCP) are generally smaller than IgG, PEG should increase their column retention to a lesser degree, whereas because DNA, endotoxin, and virus are generally larger than IgG, PEG should increase their retention to a greater degree, which should give better separation of contaminants from product. Thus, PEG can be used to dramatically enhance aggregate removal efficiency and, if desired, enhance removal of other contaminants, especially including viral particles. (Gagnon et al., “Nonionic Polymer Enhancement of Aggregate Removal in Ion Exchange and Hydroxyapatite Chromatography” presented at 12th Annual Waterside Conference, San Juan, Puerto Rico, Apr. 23-25, 2007, available at www.validated.com as Document No. PSG-070430).

### SUMMARY OF THE INVENTION

The invention provides in certain embodiments, a process for purification of a protein product from a sample comprising the protein product and aggregates of the protein product, where the process comprises the steps of (i) a first chromatography step comprising the use of a nonionic polymer for removal of the aggregates of the protein product, wherein the nonionic polymer is present at concentrations sufficient to enhance separation of the protein product from the aggregates of the protein product under the chromatography conditions, such that a fraction comprising the protein product substantially free of aggregates is collected after the step; (ii) a step of combining a solubility enhancing additive and the fraction comprising the protein product obtained in the first chromatography step or a subsequently obtained fraction comprising the protein product which fraction is derived from the fraction comprising the protein product obtained in the first chromatography step, wherein the solubility enhancing additive is selected from the group consisting of a zwitterion, a urea compound, and an alkylene glycol; and (iii) a second chromatography step comprising the use of ion exchange chromatography (or hydroxyapatite chromatography where the first chromatography step is ion exchange chromatography) wherein the solubility enhancing additive is present in sufficient concentration to enhance solubility of the protein product and substantially avoid occlusion under the chromatography conditions, wherein the solubility enhancing additive does not interfere with the second chromatography step, and wherein the process yields a purified protein product substantially free of aggregates.

### BRIEF DESCRIPTION OF THE DRAWINGS

FIG. 1 shows a reference profile for initial purification of an IgM antibody LM1 by ceramic hydroxyapatite (CUT) chromatography as described in Example 3, where total protein (A₂₈₀, A₃₀₀), turbidity (A₆₀₀), conductivity and pH were measured continuously.

FIG. 2 shows a reference profile for intermediate purification of LM1 by anion exchange chromatography as described in Example 3, where total protein (A₂₈₀, A₃₀₀), turbidity (A₆₀₀), conductivity and pH were measured continuously.

FIG. 3 shows a high-resolution reference profile of the LM1 elution peak during intermediate purification of LM1 by anion exchange chromatography as described in Example 3, where total protein (A₂₈₀, A₃₀₀), turbidity (A₆₀₀), conductivity and pH were measured continuously.

FIG. 4 shows a reference profile for polishing (final) purification of LM1 by cation exchange chromatography as described in Example 3, where total protein (A₂₈₀, A₃₀₀), turbidity (A₆₀₀), conductivity and pH were measured continuously.

FIG. 5 shows a high-resolution reference profile of the LM1 elution peak during polishing purification of LM1 by cation exchange chromatography as described in Example 3, where total protein (A₂₈₀, A₃₀₀), turbidity (A₆₀₀), conductivity and pH were measured continuously.

FIG. 6 shows a reference profile for analytical size exclusion chromatography by HPSEC of purified LM1 after polishing purification, where total protein (A₂₈₀, A₃₀₀), turbidity (A₆₀₀), conductivity and pH were measured continuously.

### DETAILED DESCRIPTION OF THE INVENTION

The present disclosure provides in certain embodiments methods and compositions for purification of a protein product through a purification process including the use of nonionic polymers in a first chromatographic separation step to enhance aggregate removal followed by an ion exchange chromatography step, wherein certain solubility enhancing additives are used at concentrations that are sufficiently high to enhance solubility of the protein product and discourage occlusion in a second chromatography step comprising ion exchange chromatography under process conditions that otherwise arc susceptible to occlusion. The labels first and second when used herein with reference to chromatography steps refer to their relative sequence but do not preclude processes involving chromatographic steps prior to the first step or between the first and second steps.

The present disclosure provides in certain embodiments methods and compositions for a multi-step process for purification of a protein product from a mixture involving a first chromatography step comprising use of a nonionic polymer and a second chromatography step involving ion exchange chromatography, wherein absent the use of a solubility enhancing additive according to the invention the protein product may form aggregates or otherwise promote occlusion during ion exchange under purification process conditions. Wherein in certain embodiments, the process includes the use of a nonionic polymer such as polyethylene glycol (PEG) in at least one step to enhance removal of aggregates from the mixture prior to the use of a solubility enhancing additive.

The solubility enhancing additive is combined with a fraction containing the protein product at a point downstream from a first chromatography step comprising use of a non-ionic polymer, such as polyethylene glycol, to promote the separation of the protein product from the aggregates of the protein product. In certain embodiments, the fraction comprising the protein product collected from the first chromatography step is collected into a composition comprising the solubility enhancing additive. In further embodiments, the fraction comprising the protein product collected following the first chromatography step is subjected to further separation or purification steps and the resulting fraction derived therefrom is then combined with a solubility enhancing additive.

Solubility enhancing additives in certain embodiments are zwitterions which promote the solubility of the protein product but have sufficiently low conductivity so as not interfere with the conduct of ion exchange chromatography. In certain embodiments, the process includes the use of zwitterions such as glycine, at concentrations sufficient to enhance solubility of the protein product and discourage occlusion under process conditions that otherwise favor aggregation or occlusion, where the zwitterion-containing compositions arc suitable for use in at least one ion exchange step, and the process yields a high-purity protein product substantially free of aggregates.

In one exemplary, non-limiting embodiment, methods and compositions are provided for use in a multi-step process for purification of IgM from a mixture, e.g., from a cell culture supernatant, wherein the process includes the use of PEG-containing buffers in at least one step that removes at least some of the IgM aggregates and provides a sample enriched in IgM (IgM monomer), the process further includes the use of low-conductivity zwitterion-containing compositions, where the zwitterions are present at concentrations sufficient to enhance IgM solubility and discourage IgM aggregate formation under process conditions that otherwise favor aggregation or occlusion in a downstream ion exchange step under process conditions, and the process includes at least one ion exchange step wherein the process yields a high-purity IgM product substantially free of aggregates.

As provided herein, the use of zwitterion-containing compositions to enhance protein solubility and avoid aggregate formation or occlusion during certain purification process steps also provides low-conductivity sample buffers that are directly compatible with ion exchange media, in contrast with the use of high-salt buffers to enhance protein solubility and avoid aggregate formation, where high-salt buffers are not directly compatible with ion exchange media. Further as provided herein, buffers containing nonionic polymers to enhance aggregate removal can be introduced directly into the zwitterion-containing compositions that enhance protein solubility and substantially avoid aggregate formation, thereby avoiding additional manipulations such as desalting, polymer removal, or buffer exchange that could affect the yield and/or quality of the purified protein product. The present methods and compositions permit compatibility between distinct orthogonal purification steps.

The present disclosure provides, in one exemplary non-limiting embodiment, methods and compositions for use in a multi-step process for purification of IgM from a cell culture supernatant, wherein the process includes the use of PEG in at least one step in a way that enhances the separation of IgM monomers from IgM aggregates and permits removal of at least some of the IgM aggregates, and the process further includes the use of low-conductivity zwitterion-containing compositions in a subsequent step, where the zwitterions are present at concentrations sufficient to enhance IgM solubility and discourage IgM aggregate formation under conditions that would otherwise favor aggregation. The process further includes an ion exchange purification step where the zwitterion-containing composition does not interfere with such ion exchange step. In one embodiment, glycine is used as the zwitterion, at concentrations sufficient to enhance IgM solubility and discourage IgM aggregate formation or occlusion under conditions that could favor aggregate formation or occlusion during ion exchange chromatography. Further, in many applications, care should be take to ensure that the purification process, once started, is completed without interruption in order to maintain enhanced solubility and reduce the risk of aggregate formation.

Solubility enhancing additives in certain embodiments arc zwitterions, urea, urea derivatives such as alkyl ureas (methyl urea, ethyl urea, etc.) or alkylene glycols such as ethylene glycol or propylene glycol. While it is believed that the mechanisms are different for different classes of solubility enhancing additives of the invention, it is believed that all enhance purification of a protein product in an ion exchange chromatographic step involving a fraction comprising the protein product and a nonionic polymer such as polyethylene glycol which is present in the protein product containing fraction as a consequence of its use in a prior chromatography step. In certain embodiments, when the solubility enhancing additive is urea, the urea may be present in concentrations up to 6 molar but preferably in concentrations below 2 molar. In certain embodiments, when the solubility enhancing additive is ethylene glycol, the ethylene glycol may be present in concentrations up to 50% but preferably in concentrations below 20%. Because excess concentrations of ethylene glycol or urea could damage some IgM antibodies, in some embodiments the concentration of the solubility enhancing additive is adjusted to approximately the minimum concentration required to avoid occlusion during the second chromatography step.

Zwitterion-Containing Compositions

Zwitterions suitable for use in the present methods and compositions, are understood to be chemical compounds that are electrically neutral, but that carry formal positive and negative charges on different atoms. Zwitterions are polar and usually have a high solubility in water and poor solubility in most organic solvents.

Glycine (Gly; G) is a small amino acid with an ionizable amino group and an ionizable carboxylic acid group. In aqueous solution at or near neutral pH, glycine will exist predominantly as its zwitterion. It is understood that the isoelectric point or isoelectric pH of glycine will be centered between the pKa values of the amino group and the carboxylic acid group in the environment in which the glycine molecule is found. It is understood that glycine has a molar dielectric increment of about 18 and that glycine should substantially enhance solvent polarity, which should in turn increase solubilizing capacity for charged molecules such as proteins. The dielectric constant of water is about 80, but for most living systems, the dielectric constant of water is about 100. The dielectric constant for 1.0 M glycine is also about 100. Glycine is a suitable zwitterion for use in the methods and compositions provided herein. Without wishing to be limited by this theory, glycine has been determined to be suitable for use in the present methods and compositions because, inter alia, glycine is zwitterionic at the pH ranges employed in the methods and compositions provided herein, such that glycine would contribute nothing to the conductivity of a solution and therefore, would not interfere with subsequent ion exchange steps. Because the buffering capacity of glycine is low to nil at the pH ranges employed in the methods and compositions provided herein, it is understood that glycine would not interfere significantly with buffer preparation. It has been observed that the effect of glycine on protein interactions with ion exchangers is nil or barely measurable, and glycine was not observed lo have any unwanted effects on practicing the purification process provided herein.

Other suitable zwitterions include, but are hot limited to, ampholytes containing both acidic and basic groups (amphoteric) that will exist as zwitterions at the isoelectric point of the ampholyte, “Good's” buffers such as the amino-sulfonic acid based buffers MES, MOPS, HEPES, PIPES and CAPS buffers, amino acid (amino-carboxylic acid) buffers such as glycine, its derivatives bicine and tricine, and alanine, buffers such as CHAPSO that can be used as detergents, and natural products including certain alkaloids and betaines.

The term “zwitterion-containing compositions” as used throughout the present specification, encompasses buffered and unbuffered solutions that contain zwitterions at a concentration sufficient to enhance protein solubility and discourage aggregate formation under conditions that would otherwise favor aggregation. The contents of zwitterion-containing compositions as provided herein, can vary depending on the intended use of the composition, where one of skill in the art can determine suitable contents for a zwitterion-containing compositions intended for a particular use. In non-limiting exemplary embodiments described in the Examples below, some zwitterion-containing compositions are unbuffered, e.g., 1.0 M glycine (unbuffered) in water at approximately pH 7 (+/−0.2), while in other exemplary embodiments, zwitterion-containing compositions include buffering agents and other components, e.g., 50 mM Tris, 1 M glycine, 2 mM EDTA, pH 8.0, or 50 mM MES, 1.0 M glycine, pH 6.2, or Buffer B: 20 mM citrate, 1.0 M glycine, pH 6.2. As illustrated by the exemplary embodiments, zwitterion-containing compositions containing sufficient zwitterions for the intended function, e.g., 1.0 M glycine, may further include zwitterionic buffering agents such as MES, or non-zwitterionic buffering agent such as Tris.

While the zwitterion-containing compositions provided herein contain zwitterions at a concentration sufficient for a particular use, it is understood that these compositions may contain zwitterions at concentration in excess of the minimum concentration necessary for a particular use. Zwitterion-containing compositions may, as a precautionary measure, contain higher zwitterion levels than the minimum needed for a particular use, without any undesirable effect. One of skill in the art can determine suitable zwitterion levels for a particular use and likewise, can determine the effects of increased or decreased zwitterion levels.

It is further understood that the use of zwitterion-containing compositions as provided herein should reduce the risk of aggregation or occlusion under ion exchange process conditions that otherwise favor aggregation or occlusion, but cannot eliminate the risk of such aggregation or occlusion. Accordingly, it may be advantageous to complete the process without interruption, in order to minimize exposure to conditions that could favor aggregation.

Purification Processes

The present disclosure provides methods and compositions for multi-step purification processes that include, but are not limited to, steps that provide sample capture, aggregate removal, and various stages of purification, where the solubility enhancing additive containing compositions are used when process conditions could favor aggregation of the protein being purified.

In particular, the present disclosure provides methods and compositions for multi-step purification processes that can be advantageously used for purification of antibodies such as IgM or IgA. Although it is understood that one of skill in the art could practice the methods and compositions provided herein to purify any protein, the non-limiting description provided below calls particular attention to the use of the present methods and composition for antibody purification. Further, although it is understood that one of skill in the art could practice the methods and compositions provided herein to purify any antibody, the non-limiting description provided below, and the exemplary embodiments provided in the Examples, particularly address the use of the present methods and composition for purification of IgMs. The non-limiting description below and in the Examples, of using the present methods and compositions for IgM purification, provides sufficient guidance and working examples to enable one of skill in the art to practice the present invention for purification of other proteins.

The present disclosure provides methods and compositions for a multi-step process of protein purification wherein the materials, reagents, and conditions for carrying out the step can be selected by one of skill in the art, depending on the conditions and circumstances of a particular application. Likewise, the present disclosure provides methods and compositions for a multi-step process of protein purification wherein the steps can be carried out in any order.

In accordance with one aspect, aggregate removal is provided wherein a solution containing the protein product, in a buffer containing a nonionic polymer such as PEG, is loaded on chromatographic media that does not operate by size exclusion, e.g., hydroxyapatite or ion exchange media, such that the protein product (monomer) can be separated from at least some of the aggregates, and a sample enriched in the protein product and substantially free of aggregates is collected. One of skill in the art can determine the optimal use of PEG-containing buffers using different chromatography media and conditions. Without wishing to be limited by this disclosure, aggregate removal using PEG-containing buffers during hydroxyapatite chromatography, especially using ceramic hydroxyapatite, was found to be reliable and easy to achieve, while aggregate removal using PEG-containing buffers during anion exchange chromatography or cation exchange chromatography was sometimes problematical and furthermore, samples eluted in PEG-containing buffers from anion exchange media or cation exchange media sometimes began to form new aggregates that required additional treatments (e.g. high salt and/or glycine) to resuspend.

In accordance with another aspect, when process conditions may favor aggregation, solutions containing the protein product also contain zwitterions at concentrations sufficient to enhance solubility of the protein product and discourage aggregate formation under aggregation-favoring process conditions such as chilling, low pH, or low conductivity. In one embodiment, a solution containing the protein product is introduced into a zwitterion-containing environment, e.g. the solution is collected into a zwitterion-containing composition having a sufficiently high concentration of zwitterions that the effectiveness of the zwitterions is maintained after dilution with the solution containing the protein product. In particular, glycine-containing compositions are suitable for use when process conditions may favor aggregation. One of skill in the art would understand that glycine can enhance protein solubility by enhancing the solvent polarity of a glycine-containing solution and thereby increasing the solubilizing capacity of the solution for charged molecules such as proteins. By way of example, polyclonal IgM solutions that arc turbid at 10 mg/ml in PBS are water-clear at 100 mg/ml in 1 M glycine. One of skill in the art would understand that because glycine is zwitterionic at the pH ranges employed in this purification process, it contributes nothing to conductivity and therefore does not interfere with subsequent ion exchange steps. Likewise, because the buffer capacity of glycine is nil within the pH range used in the present methods and compositions, glycine does not interfere significantly with buffer preparation. Finally, it is understood that, although protein interactions with ion exchangers are slightly weaker in solvents with high dielectric constants, because the ion exchange groups have to compete with the solvent, it has been observed that the effect of glycine, with a dielectric constant of about 100, on protein interactions with ion exchangers, is barely measurable in diverse exemplary embodiments, such that glycine was not observed to have any practical effect on the present purification process. Glycine can be used as the solubility enhancing additive as provided herein, in concentrations ranging between about 50 nM to about 5 M, or between about 100 mM to about 4 M, or between about 250 mM to about 3 M, or between about 500 mM to about 2 M, or between about 750 mM to about 1 M. Glycine can be used in solutions of about 50 mM, or about 100 mM, or about 250 mM, or about 500 mM, or about 750 mM, or about 1 M, or about 1.1 M, or about 1.2 M, or about 1.3 M, or about 1.4 M, or about 1.5 M, or about 1.6 M, or about 1.7 M, or about 1.8 M, or about 1.9 M, or about 2 M. It is understood that glycine can be used at concentrations higher than the concentration necessary to achieve a desired effect, e.g., to enhance protein solubility and/or to avoid aggregate formation, as a precautionary measure, where one of skill in the art can determine the glycine concentrations that can be tolerated in a particular application.

Protein product purification as provided herein yields a purified protein product substantially free of aggregates. The aggregate content of a purified protein sample substantially free of aggregates, as provided herein, can be less than about 5%, and is expected to be less than about 1%, or less than about 0.5%, or less than about 0.1%, and may be below the detection limit of the method being used to measure aggregate content. In particular, the aggregate content of a purified IgM sample substantially free of aggregates can be less than about 5%, and is expected to be less than about 1%, or less than about 0.5%, or less than about 0.1%, and may be below the detection limit of the method being used to measure aggregate content.

Protein product purification as provided herein can be carried out using linear gradients, step gradients, or a combination linear and step gradients for product separation and recovery. In accordance with one aspect, a linear gradient may be used to achieve better separation of the protein product from aggregates and/or from other contaminants such as HCP. In accordance with another aspect, a step gradient may be used to reduce the volume of eluted product. The choice of linear and/or step gradients to reach the same endpoint is made with the understanding that either choice could produce a subtle shift of selectivity that could affect purity and aggregate content. The choice of a step and/or linear gradient is made with the understanding that the setpoints for step intervals are partly a function of column loading, where the setpoints for a column loaded to 95% of breakthrough capacity are significantly lower than the setpoints for a column that is loaded to 50%. Selection and use of a gradient for a particular embodiment can be performed using factors and methods known to one of skill in the art.

Initial Purification

A first step is carried out to accomplish initial purification, yielding a fraction enriched in the protein product. When initial purification includes sample capture, the enriched fraction collected after initial purification is expected to have a higher concentration of protein product than the starting material. When initial purification does not include sample capture, the enriched fraction may not have a significantly higher concentration of protein product, but will nonetheless be enriched in the protein product due to separation from at least some contaminants in the starting material (e.g., when the starting material is passed over media that binds certain contaminants and docs not bind the protein product). When initial purification includes aggregate removal, the enriched fraction is expected to be substantially free of aggregates. When initial purification does not include aggregate removal, aggregates will be removed in another purification step. In one embodiment, a first step accomplishes sample capture, aggregate removal, and initial purification, yielding a fraction highly enriched in protein product and substantially free of aggregates, where the concentration of protein product is higher than in the starting material. In another embodiment, a first step accomplishes sample capture and initial purification, but does not include aggregate removal, yielding a fraction having a concentration of protein product that is higher than in the starting material, where the fraction is enriched in protein product due to separation of the protein product from at least some contaminants, and the fraction contains aggregates formed prior to and/or during the first step. Optionally, if it is expected that the process conditions favor aggregation, initial purification may be carried out using zwitterion-containing compositions.

Intermediate Purification

Another step is carried out to accomplish intermediate purification, yielding a protein product fraction that is even more highly enriched in the protein product than the fraction collected after initial purification. If initial purification did not include sample capture, then sample capture to increase the concentration of protein product can be carried out during intermediate purification. If initial purification did not include aggregate removal, then aggregate removal can be carried out during intermediate purification. In one embodiment, after initial purification including sample capture and aggregate removal, the more concentrated and substantially aggregate-free protein product fraction is further purified by ion exchange, e.g., anion exchange or cation exchange, yielding a concentrated, substantially aggregate-free protein product fraction of higher purity. In another embodiment, after initial purification including sample capture, the concentrated protein product fraction is further purified by intermediate purification including aggregate removal, yielding a concentrated, substantially aggregate-free protein product fraction of higher purity. In another embodiment, after initial purification to separate the protein product from certain contaminants, the protein product fraction is further purified, including sample capture and aggregate removal, yielding a concentrated, substantially aggregate-free protein product fraction of higher purity. Intermediate purification may, or may not, be carried out in the presence of zwitterion-containing compositions, where it is understood that if the process conditions favor aggregation, intermediate purification will be carried out using zwitterion-containing compositions.

Final, Polishing Purification

A further step is carried out to accomplish final, or “polishing” purification of the protein product. It is expected that the protein product fraction collected after this has a purity in excess of 99%, with no detectable contaminants or aggregates. Polishing purification may, or may not, be carried out in the presence of zwitterion-containing compositions or other solubility enhancing additives, where it is understood that if the process conditions favor aggregation, polishing purification will be carried out using zwitterion-containing compositions. Polishing purification can be carried out using any suitable method, including but not limited to, hydroxyapatite chromatography or ion exchange chromatography.

Additional Steps

Purification processes as provided herein may include additional steps including, but not limited to, filtration, virus inactivation (e.g., by the solvent/detergent (S/D) method) or additional contaminant removal steps. Although the process may include optional filtration, desalting, diafiltration, or buffer exchange steps, the methods and compositions provided herein arc expected to reduce or eliminate many such steps. Analytical measurements may be made al any time during the process, e.g., to evaluate the sample purity and aggregate content of samples collected at multiple stages, to determine the effect of various process parameters. Purity of the IgM fractions collected after polishing purification can be evaluated by various analytic measurements such as analytical SEC (e.g., HPSEC as in Example 4), electrophoretic measurements (e.g., denaturing or non-denaturing gel electrophoresis, 1EF, 1-D or 2-D electrophoresis, etc.), peptide fingerprinting (GC-MS, Maldi-TOF, etc.) In accordance with one aspect, analytical SEC (e.g., HPSEC) of the protein product fraction after polishing purification can be carried out to verify that the protein product has a purity in excess of 99% and is free of detectable contaminants.

Sequence of Process Steps

The sequence of process steps as provided herein can follow any order, as long as precautions are taken to achieve aggregate removal using nonionic polymers and to use solubility enhancing additives such as zwitterion-containing compositions as necessary to maintain enhanced solubility and provide compatibility between chromatographic modes. In exemplary embodiments described in the Examples below, the first purification step involves sample capture, aggregate removal, and initial purification on ceramic hydroxyapatite (CHT) in the presence of PEG (for aggregate separation and removal), where fractions from CHT are collected into a zwitterion-containing composition that will be compatible with the next step of intermediate purification on anion exchange media, followed by a polishing purification step on cation exchange media. Purification in accordance with the present invention can be carried out using a different sequence of steps. In another embodiment, the first step involves sample capture and initial purification on cation exchange, followed by intermediate purification and aggregate removal on CHT (with PEG), and a final step of polishing purification by anion exchange. In yet another embodiment, the first step involves sample capture and initial purification on anion exchange, followed by intermediate purification and aggregate removal on CHT (with PEG), and a final step of polishing purification by cation exchange. In yet another embodiment, the first step involves sample capture and initial purification on cation exchange, followed by intermediate purification by anion exchange, followed by aggregate removal and a final step of polishing purification on CHT. In yet another embodiment, the first step involves sample capture and initial purification by anion exchange, followed by intermediate purification by cation exchange, followed by aggregate removal and a final step of polishing purification on CHT.

In certain embodiments, the first chromatography step comprises cation exchange chromatography including polyethylene glycol in amounts sufficient for aggregate removal and the second chromatography step comprises hydroxyapatite chromatography or anion exchange chromatography. In certain other embodiments, the first chromatography step comprises anion exchange chromatography including polyethylene glycol in amounts sufficient for aggregate removal and the second chromatography step comprises hydroxyapatite chromatography or cation exchange chromatography.

Purification of IgM Antibodies

The present disclosure provides particular methods and compositions that can be advantageously used for purification of IgM. Certain characteristics of IgMs allow the development and use of orthogonal purification procedures under conditions that can achieve significant IgM purification in few steps, thereby eliminating unnecessary steps that could reduce the yield and/or purity of the recovered IgM. For example, most IgMs (including monoclonal IgMs) are highly charged and therefore, are retained strongly enough by ion exchangers to support high binding capacities at moderate pH values. In addition, IgMs bind strongly to hydroxyapatite at physiological values of pH and conductivity, which favors the use of hydroxyapatite in IgM purification.

Thus, methods and compositions are provided to use certain characteristics of IgMs that may be advantageous for purification, and to reduce or avoid problems that may arise during purification due to certain characteristics of IgMs. For example, IgM purification will differ from IgG purification, given that IgMs tend to be soluble in a narrower range of conditions than IgGs, IgMs are more susceptible to denaturation than IgGs, IgMs often denature upon exposure to hydrophobic surfaces (e.g., in hydrophobic interaction chromatography), and IgMs are sensitive to pH extremes and tend to precipitate under conditions that are routinely used for anion exchange or affinity purification of IgGs, where low conductivity solutions tend to compound the pH sensitivity of IgMs. Thus in certain embodiments the use of solubility enhancing additives inhibits occlusion during ion exchange chromatography.

The present methods and compositions provide IgM purification processes that include the use of PEG-containing solutions to enhance removal of IgM aggregates from a complex mixture such as a cell culture supernatant, and further include the use of zwitterion-containing compositions (e.g. containing glycine at about 1.0 M) during ion exchange chromatography, to enhance IgM solubility and stabilize IgM under conditions that could otherwise favor aggregation, with the goal of avoiding or at least reducing formation of new aggregates during the IgM purification process.

Non-limiting exemplary embodiments of the present methods and compositions arc presented in the Examples below. In the Examples, three different monoclonal IgMs—SAM6, CM1, and LM1—are purified as provided herein. In the embodiments described below, the following purification steps are practiced: (I) sample capture and initial purification by hydroxyapatite chromatography in the presence of PEG-containing buffers; (II) intermediate purification by anion exchange chromatography in the presence of zwitterion-containing compositions; and (III) polishing purification by cation exchange chromatography in the presence of zwitterion-containing compositions to yield highly purified IgM that is substantially free of aggregates. The sequence of purification steps as provided herein can follow any order, as long as the process is practiced in a way that accomplishes the removal of aggregates and the use of zwitterion-containing compositions to maintain enhanced IgM solubility and to avoid IgM aggregation. In accordance with another aspect presented herein, the sequence of purification steps may be carried out in a way that provides buffer compatibility between different chromatographic modes in different steps.

Initial Purification of IgM from Cell Culture

In exemplary embodiments presented in the Examples below, hydroxyapatite chromatography in the presence of PEG-containing buffers is used for sample capture, aggregate removal, and an initial purification step, yielding a fraction highly enriched in IgM and substantially free of aggregates, where the IgM-containing fraction is then introduced into a zwitterion-containing composition. In the presence of PEG, IgM (monomer) and IgM aggregates bind to hydroxyapatite, but have different elution profiles due to the size-selective effect of PEG as a buffer additive. Ceramic hydroxyapatite (CHT) is suitable for this step.

It is understood that extensive washing after sample loading is important to achieve optimal purification performance from this step. After a sample has been loaded and the column (media) extensively washed, the sample is eluted by increasing the salt concentration to a predetermined level, by a linear gradient or by a step gradient, alter which time the column is held at that salt concentration until the antibody peak has eluted. By way of example, IgM can be eluted from CHT using a linear gradient from 125 mM to 350 mM sodium phosphate over 5 CV (Example 1, 25% to 70% Buffer B), or by a linear gradient from 165 mM to 365 mM sodium phosphate over 5 CV (Example 2, 33% to 73% Buffer B) or by a linear gradient from 100 mM to 325 mM sodium phosphate over 5 CV (Example 3, 20% to 65% Buffer B). All buffers were at pH 7.0 and contained 10% PEG-600.

During elution from hydroxyapatite, sample purification can be enhanced by collecting fractions from the center of the IgM elution peak, according to a strategy that is expected to exclude early-eluting contaminants on the leading side of the elution peak and, more importantly, is expected to exclude aggregates eluting later than IgM, on the trailing side of the IgM elution peak. As described below, the IgM elution peak can be collected directly into zwitterion-containing composition, e.g., 1 M glycine. As the presence of aggregates can cause turbidity, the “water-clear” IgM elution peak appeared to be largely aggregate-free, and the IgM fraction remains clear after being collected into 1 M glycine. The linear gradient segment can be converted to a step gradient to reduce eluted product volume.

Sample purity after the initial purification step (e.g., IgM content of the pooled IgM elution peak fractions, as % of total protein) can be in excess of about 50%, or in excess of about 60%, or in excess of about 70%. or in excess of about 80% or in excess of about 85%, or in excess of about 90%, or in excess of about 95%. One of skill in the art can measure the sample purity after this step for a particular application and, if desired, alter process conditions to improve sample purity. In the exemplary embodiments below, the purity of the SAM6 sample after CHT was in excess of 90%, possibly in excess of 95% (Example 1), and the purity of the LM1 sample after CHT was in as high as 90%. In the exemplary embodiment in Example 2 below, the purity of the CM1 sample after CHT only appeared to be about 50%, but this was considered acceptable given that contaminants were easily eliminated in the following anion exchange step.

When the initial purification step is hydroxyapatite chromatography in the presence of PEG-containing buffers, this step provides the major aggregate removal step. The aggregate content (measured as % of total protein by analytical size exclusion chromatography) of a protein sample can be less than about 5%, and is expected to be less than about 1%. In particular, the aggregate content of an IgM sample can be less than about 5%, and is expected to be less than about 1%. If the aggregate content is greater than about 1%, one of skill in the art can alter elution conditions to achieve better separation of IgM from aggregates, e.g. by lowering final salt concentration for elution from hydroxyapatite. In certain embodiments, the presence of aggregates was undetectable by analytical size exclusion chromatography on G4000SWXL, where the limit of detectability is assumed to be about 0.1%, such that lack of detectable aggregates is generally interpreted to indicate that aggregate content is below 0.1%. When IgM fractions from subsequent purification steps arc analyzed, aggregates are entirely or mostly absent, which suggests that aggregates found in the starting material are produced during cell culture. This result is consistent with the pattern of aggregate formation seen for IgGs. However, after this step, conditions must be avoided that could result in the formation of new aggregates during the remainder of the purification process. Thus, zwitterion-containing compositions are to be used to enhance IgM solubility and avoid aggregate formation during the remainder of the purification process.

One of skill in the art can identify PEG polymers and concentrations that would be suitable for this step. In the non-limiting embodiments described below, PEG-600 and PEG-1000 can be used interchangeably, at the same concentration. It has been observed that the effect of PEG-1000 is slightly stronger, which will cause the antibody to elute a little later and will similarly enhance removal of aggregate. PEG can be omitted entirely, which is likely to result in IgM eluting earlier, with the salt concentration of wash and elution buffers adjusted accordingly.

A zwitterion level of 1 M glycine may be higher than is necessary and as such, may be considered precautionary. Although it may be possible to reduce the glycine level without risk to the yield and/or purity of the IgM product, the effects of reducing zwitterion levels should be verified experimentally before glycine levels are reduced, both for preparative and for large-scale purifications.

As noted below, virus inactivation by the solvent/detergent (S/D) method can be performed during this step, while the antibody is bound to CHT or after elution from CHT.

II. Intermediate Purification of IgM by Anion Exchange Chromatography

In exemplary embodiments presented in the Examples below, anion exchange in the presence of zwitterion-containing compositions can be carried out to further purify the IgM sample in an intermediate purification step. In the exemplary embodiments, because aggregate removal was accomplished using PEG-containing buffers during initial purification on CHT, solutions in the following purification steps do not contain PEG but they do contain zwitterions (glycine) at concentrations sufficient to enhance IgM solubility and avoid aggregate formation. It is recommended that intermediate purification of IgM by anion exchange chromatography commence as soon as possible after the initial purification on CHT, e.g., within 24 hours of completing the initial purification on CHT.

In the exemplary embodiments, the pH of the sample solution (pooled fractions from IgM eluate peak collected from CHT collected into 1M glycine) was adjusted to a suitable high pH (Tris 50 mM, pH 8.0) and loaded on anion exchange media, e.g., a quaternary amine strong anion exchanger such as CIM® QA (CIM® Convective Interaction Media, BIA Separations, Klagenfurt, Austria). Other anion exchange media can be used, including weak ion exchangers such as DEAE or EDA which may have higher capacity than QA, although differences in selectivity and buffering effects on weak anion exchangers may require adjustments such as more extensive column equilibration, and may diminish pH control during elution. Anion exchangers in monolith form, as illustrated in the Examples below, can be used if available, although non-monolith anion exchangers can also be used, where process parameters such as flow rates will be adjusted, and possible reductions in capacity and contaminant removal, especially virus removal, will be taken into consideration. Although steps can be performed in any order, carrying out anion exchange chromatography as a second step can be advantageous when the sample elutes from the first step at a high salt concentration (e.g., IgM elutes from CHT at a high salt concentration) because anion exchange is more salt-tolerant than cation exchange, such that fractions eluted from CHT at relatively high salt concentrations would not present compatibility problems with anion exchange, especially after substantial dilution during sample loading.

Sample containing IgM can be loaded on the column by in-line dilution, which avoids exposing IgM to sudden changes in pH, buffer composition, or salt levels, that could favor aggregation (denaturation). In exemplary embodiments presented in the Examples, in-line dilution of 1 part sample containing IgM supplied by one pump, to 2 parts loading buffer supplied by a different pump, resulted in a total dilution of 10× the volume of the IgM fraction eluted from the CHT column. Other in-line dilutions or different sample loading techniques could be used to introduce the sample for intermediate purification.

After the sample is loaded, the column is extensively washed, which may elute a small peak of material. IgM is eluted by increasing the salt concentration to a predetermined level, by a linear gradient or by a step gradient, after which lime the column is held al that salt concentration until the antibody peak has eluted. In the exemplary embodiments in the Examples, NaCl gradients eluted SAM6 at about 200 mM NaCl, 0.5 M glycine (Example 1) and CM1 at about 225 mM NaCl, 0.5 M glycine (Example 2), and a sodium phosphate buffer elutes LM1 at about 250 mM sodium phosphate, 0.5 M glycine. Fractions are collected beginning at 10% of maximum peak height on the leading edge, until 10% of maximum peak height on the trailing edge, and the collected fractions are pooled. It was expected that any remaining aggregate eluted on the trailing side. Due to the low pH (6.2) of the elution buffer, it is recommended that the pooled fractions containing IgM be held for less than about 24 hours after this step. Anion exchange can be completed in less than an hour, but can be slowed down for convenience, as it is understood that reducing flow rate will neither improve column performance nor diminish it. If a viral filtration step is anticipated and has not been carried out previously, viral filtration could optionally be carried out after intermediate purification by anion exchange.

Polishing Purification of IgM by Cation Exchange Chromatography

Pooled fractions after intermediate purification are then subjected to a final or “polishing” purification. In the exemplary embodiments presented in the Example, pooled IgM-containing fractions eluted from intermediate purification by anion exchange chromatography were further purified by cation exchange chromatography using zwitterion-containing compositions. Given the relatively low conductivity (low salt concentration) of the initial buffers, the use of zwitterion-containing compositions, e.g. 1 M glycine, is important to maintain protein solubility and avoid aggregate formation under cation exchange conditions. Suitable media include the sulphonic strong cation exchanger CIM® SO3 (monolith), or other strong or weak cation exchange media, in various formats, as can be selected and used by one of skill in the art to practice the present methods and compositions.

After the sample is loaded, the column is extensively washed. Issues of buffer exchange and column compatibility can be avoided by in-line dilution (e.g., 10×) of the IgM sample in cation exchange column equilibration buffer containing 1M glycine.

IgM is eluted by increasing the salt concentration to a predetermined level, by a linear gradient or by a step gradient, after which time the column is held at that salt concentration until the antibody peak has eluted. High-purity IgM is recovered by collecting fractions beginning at 10% of maximum peak height on the leading edge, until a predetermined cutoff point in the trailing edge, and pooling the collected fractions. If any aggregate was present in the solution, it is expected that any remaining aggregate would elute on the trailing side of the IgM peak. Cutoff points for collecting IgM fractions on the trailing edge of the IgM peak can be at 40% of maximum peak height, or 30% of maximum peak height, or 25% of maximum peak height, or 20% of maximum peak height, or 15% of maximum peak height, or 10% of maximum peak height.

Recovery efficiency for this step can be in excess of about 75%, or in excess of about 80%, or in excess of about 85%, or in excess of about 90%, or in excess of about 95%, of the total detectable IgM applied to the column. Purity of the IgM fractions collected after polishing purification can be evaluated by various analytic measurements, e.g. by HPSEC as in Example 4. Purity after polishing purification can be in excess of about 80%, in excess of about 90%, in excess of about 95%, or in excess of about 99%. The final IgM preparation is expected to be free of detectable aggregates (i.e., if any aggregates arc present, they are present in quantities that are below the limits of detection).

It is understood that when a cation exchange step is performed, the cation exchange step may be the most critical step in the entire process with respect to avoiding aggregate formation, as cation exchange exposes the antibody to conditions that favor aggregation, including low pH and low conductivity. Although high glycine levels arc very important to maximize solubility, it is further understood that high levels of glycine or another suitable zwitterion reduces the risk of aggregation but does not eliminate it. Methods and compositions as provided herein provide additional measures to avoid unwanted aggregation formation. For example, interruptions during cation exchange should be avoided, taking care to ensure that the cation exchange process, once started, is completed without interruption.

Unless defined otherwise, technical and scientific terms used herein have the meaning commonly understood by a person skilled in the art to which this invention belongs. As used herein, the following terms have the meanings ascribed to them unless specified otherwise.

As used herein, the singular forms “a,” “an,” “the,” and “is” include plural referents unless the context clearly indicates otherwise. Thus, for example, reference to “a compound” includes a plurality of compounds and reference to “a residue” or “an amino acid” includes reference to one or more residues and amino acids.

All publications, patents, and patent applications cited herein, are hereby expressly incorporated by reference for all purposes.

### EXAMPLES

### Example 1

### Purification Procedure for SAM6

I. Sample Capture and Initial Purification by Hydroxyapatite Chromatography.

SAM6 IgM was purified from a starting material of one liter of clarified cell culture supernatant, containing approximately 200 μg IgM/ml of cell culture supernatant. First, cell culture supernatant was filtered using a 0.22 micron (0.22 μm) filter, and followed by addition of 500 mM Na phosphate, pH 7.0, at 1% v:v, to yield a final phosphate Concentration of 5 mM. If the sample already contained phosphate, then the minimum amount of 500 mM Na phosphate, pH 7, necessary to yield a phosphate concentration of at least 5 mM was added to the filtered supernatant. A solution of 1 M Tris, pH 8.0, was added at 1% v:v, to yield a final concentration of 10 mM Iris, which was expected to yield a final pH of 6.8 to 7.2. The sample solution was allowed to reach room temperature (18-23° C.).

Conditions and Reagents for Hydroxyapatite Chromatography
Media/column: CHT type II, 40 micron, ATOLL 11.3×100 mm column
Flow rate: 100-200 cm/hr (1.67-3.34 ml/min on Atoll column)
Buffer A: 10 mM sodium phosphate, 10% PEG-600, pH 7.0
Buffer B: 500 mM sodium phosphate, 10% PEG-600, pH 7.0
Buffer C: 1.0 M glycine (unbuffered) pH 7 (+/−0.2)
Buffer D: 600 mM KPO4, pH 7
Buffer E: 1.0 M NaOH
Buffer F: 0.1 M NaOH, or 20% ethanol, 5 mM sodium phosphate pH 7

### Hydroxyapatite Chromatography.

The column (ceramic hydroxyapatite CHT™ type II 40 micron (Bio-Rad Laboratories, Hercules, Calif.), 11.3×100 mm column pre-packed by ATOLL Gmbh) was equilibrated in Buffer A (above). The sample was applied in 100 column volumes (100 CV) of Buffer A. After the sample was loaded, the column was washed with between 2 to 5 CV Buffer A (Wash 1). The column was then washed with 25% buffer B (125 mM phosphate, 10% PEG-600) until readings returned to baseline values as determined by measuring absorption at 280 nm, A280 (Wash 2).

Sample was eluted from the column with a one (1) CV linear gradient to 70% Buffer B (350 mM phosphate, 10% PEG-600), and the column was then held at 70% Buffer B until the product peak eluted. Fractions of 0.5 CV were collected directly into 1.15 CV of 1 M glycine (Buffer C). The eluting peak was water-clear, and remained so when diluted with glycine. Fractions were stored at 4° C. As recommended, fractions from 10% of maximum peak height on the leading side, to 10% maximum peak height on the trailing side were pooled for further purification. It was expected that this collecting/pooling strategy excluded aggregates that may begin to elute on trailing side of the sample peak. The CHT column was cleaned with 5-10 CV Buffer D, sanitized with Buffer E, and stored in Buffer F.

Initial purification on CHT required about 6 hours, at a flow rate of 20 mg/hr for a 10 cm bed height, which included about 5 hours for sample loading. Sample purity was in excess of 90% IgM, with an aggregate level of less than 1%

Comments on Initial Purification

Preliminary data suggested that most of the IgM was bound when 100×volume was applied to 1×volume of CHT. If significant product losses are detected in the later flow-through fractions, then the sample application volume should be reduced accordingly. It was calculated that process time will increase with bed height, such that doubling bed height will double process time, It was therefore concluded that under these conditions, a 15 cm bed at full process scale is adequate, and 10 cm bed may be adequate, depending on the ability to consistently obtain good packing quality, and a 20 cm bed at full process scale should not be exceeded.

Extensive washing after sample loading is important to achieve optimal purification performance from this step. When a large peak eluted in this wash, possibly as much as twice the size of the later IgM elution peak, suggesting apparent product losses up to 5%, this peak was likely to contain various contaminants such as host cell proteins (HCP), as well as IgM fragments, which were often still detectable by anti-IgM antibodies that cannot discriminate between intact IgM and fragments. Although the salt concentration of the wash buffer could be lowered to prevent apparent IgM loss, this may increase contamination by HPC.

If desirable or necessary, virus inactivation by the solvent/detergent (S/D) method can be performed during this step, while the antibody is bound to CHT or after elution from CHT. If performed while the antibody is bound to CUT, then it should be done after the first wash (Buffer A). In one method CV of S/D reagent is prepared according to methods known in the art, and a first CV of S/D reagent is rapidly passed over the column (200 cm/hr), after which the second CV of S/D reagent is slowly passed over the column for an hour. The column is washed with at least 10 CV of 10 mM phosphate+the detergent used in the S/D step, to remove residual 2-percent tri(n-butyl)phosphate (TNBP), and then washed with 5 CV Buffer A to remove residual detergent and recommence the purification. S/D treatment may alternatively be performed after the CHT step, since it is also compatible with the following anion exchange step. Note that the effects of S/D treatment on this antibody have not been evaluated.

II. Intermediate Purification by Anion Exchange Chromatography.

Intermediate purification by anion exchange chromatography was commenced within 24 hours of completing the initial purification on CHT.

Conditions and Reagents for Anion Exchange Chromatography
Media/column: CIM® QA monolith (8 ml)
Flow rate: up to 10 CV per minute.
Buffer A: 50 mM Tris, 1 M glycine, 2 mM EDTA, pH 8.0
Buffer B: 50 mM MES, 10 mM NaCl, 1.0 M glycine, pH 6.2
Buffer C: 50 mM MES, 500 mM NaCl, pH 6.2
Buffer D: 1.0 M NaOH
Buffer E: 0.01 M NaOH or 20% ethanol

Anion Exchange Chromatography

A solution of 1 M Tris, pH 8.0 was added to the pooled fractions collected from CHT, at 5% v:v, to yield a final Tris concentration of 50 mM, and the sample solution was allowed to reach room temperature (18-23° C.).

The column containing eight (8) ml strong anion exchanger CIM® QA monolith was equilibrated in Buffer A, and the sample solution was loaded on the column by in-line dilution as follows: 1 part sample (supplied by Pump A) to 2 parts Buffer A (supplied by Pump B). This sample dilution resulted in a total dilution of 10×the volume eluted from the CHT column. The expected column capacity of the QA monolith used for this step was about 30 mg IgM per ml of monolith. The column was washed with Buffer B (Wash 1) and then washed with 77% Buffer B, 23% Buffer C (wash 2). which eluted a small peak. If desired, Buffer C can be formulated to contain 1M NaCl to provide more effective cleaning, although gradient setpoints are adjusted accordingly.

Sample was then eluted using a 5CV linear gradient to reach 52% Buffer B, 48% Buffer C, and the column was held at 52% Buffer B, 48% Buffer C until the sample peak was fully eluted. The sample peak containing IgM eluted at about 200 mM NaCl and 0.5 M glycine, and was clear. Fractions collected beginning at 10% of maximum peak height on the leading edge until 10% of maximum peak height on the trailing edge, were pooled. It was expected that any remaining aggregate eluted on the trailing side. The column was cleaned with 100% Buffer C, which produced a small sharp peak containing a small account of IgM mixed with several contaminants, followed by a succession of other small contaminant peaks. The column was sanitized with Buffer D and stored in Buffer E. This intermediate purification step was completed in less than one hour.

In Buffer A, EDTA was expected to remove any calcium that may have been picked up by the sample during the CHT step, and pH 8.0 was used to enhance binding capacity of the media. Wash and elution were carried out al 6.2 to enhance removal of host cell protein (HCP), and to provide eluted sample at a pH that will be directly compatible with buffers using in the following cation exchange purification. Due to the low pH (6.2) of the elution buffer, the pooled fractions containing IgM were held for Jess than about 24 hours after this step.

Comments on Intermediate Purification

If a viral filtration step is anticipated and has not been carried out previously, it can be carried out after the anion exchange step, in which case a chase solution of 50 mM MES, 150 mM NaCl, pH 6.2 should be used, and the antibody should be re-concentrated during the following cation exchange step.

If virus inactivation by the solvent/detergent (S/D) method was applied after the CHT step (see Comments above), then an additional detergent wash should be applied to the anion exchange process, e.g., by adding detergent to anion exchange Buffer A and applying at least 10CV of Buffer A after sample application. In this case, purification is recommenced at the Buffer B wash.

The strong binding of this antibody to anion exchangers suggests that the elution pH could be reduced further; however this increases the risk of product denaturation, and although high glycine solutions will reduce this risk, they cannot eliminate it.

III. Polishing Purification by Cation Exchange Chromatography.

Cation exchange chromatography was commenced within 24 hours of the anion exchange step (above).

Conditions and Reagents for Cation Exchange Chromatography
Media/column: CIM® SO3 monolith (8 ml)
Flow rate: up to 10 CV per minute.
Buffer A: 50 mM MES, 1.0 M glycine, pH 6.2
Buffer B: 20 mM citrate, 1.0 M glycine, pH 6.2
Buffer C: 250 mM citrate, pH 6.2
Buffer D: 1.0 M NaOH
Buffer E: 0.01 M NaOH or 20% ethanol

Cation Exchange Chromatography

Once started, the final purification step using cation exchange chromatography was completed without interruption.

Sample (pooled fractions from anion exchange) was allowed to reach room temperature (18-23° C.). The column was equilibrated in Buffer A. Sample was loaded by in-line dilution of 1 part sample solution to 9 parts Buffer A. Capacity of the CIM SO3 media appeared to be about 30 mg IgM/ml. The column was washed with 2-5 CV Buffer A (Wash 1: 2 CV is sufficient, no more than 5 CV), and then washed with 5CV 95% Buffer B, 5% Buffer C (Wash 2) which produced a small peak.

Sample was eluted using a 5 CV linear gradient to reach 60% Buffer B, 40% Buffer C, and the column was then held at 60% Buffer B, 40% Buffer C the sample peak was fully eluted. Fractions collected beginning at 10% of maximum peak height on the leading edge until 10% of maximum peak height on the trailing edge, were pooled. IgM eluted clear. It was expected that any remaining aggregate eluted on the trailing side. A solution of 500 mM phosphate pH 7 was added to the pooled fractions at 10% v:v, to raise the pH, and the solution was stored at 4° C. The column was cleaned with Buffer B, which produced a small peak. The column was sanitized in Buffer D and stored in Buffer E.

Comments on Polishing Purification

The cation exchange step may be the most critical step in the entire process because it exposes the antibody to conditions that favor aggregation, including low pH and low conductivity. Although high glycine levels are very important to maximize solubility, this only reduces risk but does not eliminate it. Interruptions should be avoided, such that care must be taken to ensure that the cation exchange process, once started, is completed without interruption.

### Example 2

### Purification Procedure for CM1

I. Capture and Initial Purification by Hydroxyapatite Chromatography

CM1 IgM was purified from a starting material of 500 ml of clarified cell culture supernatant, with approximately 200 μg IgM/ml of cell culture supernatant. First, cell culture supernatant was allowed to reach room temperature (18-23° C.) and then filtered suing a 0.22 microns (0.22 μm) filter, followed by addition of 500 mM Na phosphate, pH 7.0, at 1% v:v, to yield a final phosphate concentration of 5 mM. If the supernatant already contained phosphate, then the minimum amount of 500 mM Na phosphate, pH 7.0, necessary to yield a phosphate concentration of at least 5 mM was added to the filtered supernatant.

Conditions and Reagents for Hydroxyapatite Chromatography
Media/column: CHT type II, 40 micron, ATOLL 11.3×100 mm column
Flow rate: up to 200 cm/hr (3.34 ml/min on ATOLL column)
Buffer A: 10 mM sodium phosphate, 10% PEG-600, pH 7.0
Buffer B: 500 mM sodium phosphate, 10% PEG-600, pH 7.0
Buffer C: 1.0 M glycine (unbuffered) pH 7 (+/−0.2)
Buffer D: 600 mM KPO₄, pH 7
Buffer E: 1.0 M NaOH
Buffer F: 0.1 M NaOH, or 20% ethanol, 5 mM sodium phosphate pH 7

Hydroxyapatite Chromatography

The column (ceramic hydroxyapatite CHT type II, 40 micron, 11.3×100 mm column, ATOLL Gmbh) was equilibrated in Buffer A. Sample solution was applied in 50 column volumes (CV). After the sample was loaded, the column was then washed with 2-5 CV Buffer A (Wash 1: 2 CV is sufficient; no more than 5 CV is necessary; no need to wash to baseline.) The column was then washed with 23% Buffer B (165 mM phosphate, 10% PEG-600), until readings returned to baseline (Wash 2). A large peak eluted in the second wash step, roughly equivalent to the product peak, where IgM fragments were expected to be eluted by this wash. As noted above, apparent product losses in the range of 5-10% are likely to be fragments displaced by this wash; if losses of intact product seem excessive, the concentration of buffer B could be reduced, but this will probably increase contamination by HCP.

Sample was eluted from the column with a 5 CV linear gradient to reach 73% Buffer B (365 mM phosphate, 10% PEG-600), after which point the column was held al 73% Buffer B until the product peak eluted. Fractions of 0.5 CV were collected directly into 1.15 CV of 1 M glycine (Buffer C). The eluting peak was water-clear, and remained so when diluted with glycine. Fractions from 10% of maximum peak height on the leading side, to 10% maximum peak height on the trailing side, were pooled for further purification. It was expected that this collecting/pooling strategy excluded aggregates that may begin to elute on the trailing side of the peak. Fractions were stored al 4° C. The CHT column was cleaned with 5-10 CM Buffer D, sanitized with Buffer E, and stored in Buffer F.

Initial purification on CHT required about 3.5 hours, at a flow rate of 200 cm/hr for a 10 cm bed height, which includes about 2.5 hours required for sample loading. Sample purity after CHT, based on anion exchange results, was about 50%. This sample purity was substantially lower than for SAM6 (Example 1 above) or LM1 (Example 3 below), but the contaminants were easily eliminated in the following anion exchange step. It was established that the CHT step was the major aggregate removal step in this process, as aggregates were undetectable by analytical size exclusion chromatography on G4000SWXL (data not shown), which was interpreted to indicate that aggregate content was below 0.1%. Total recovery, compared with the initial sample loaded on the column, was low, largely due to elimination of IgM fragments in the wash steps and elimination of aggregates in the cleaning step.

Comments on Initial Purification

The binding capacity of the CHT step may be the least defined parameter of the purification process. Preliminary data suggested that most of the IgM is bound when a 50×sample volume is applied to a 1×volume of CHT. The strong binding of CM1 to CHT (stronger than both LM1 and SAM6) suggested that substantially higher column capacity should be possible, but competition by a major contaminant (described below) may be a limitation. As usual for any application, flow-through fractions were retained during the first few runs and tested for IgM content so that column capacity could be verified. When efficient binding was confirmed, then the loading volume could be increased. If significant product losses were detected in the later flow-through fractions, then the sample application volume was reduced accordingly. Likewise, the unpredictability of column life led to the suggestion to prepare dedicated columns for the CHT step, designed to accommodate the high density and settling rates of CHT, where the dedicated column should never be unpacked unless required by introduction of air or cumulative loss of performance.

As expected, recovery was lowest at this step, due in particular to elimination of fragments (in the wash) and aggregates (in the cleaning step). Given a 90% recovery from both the following ion exchange steps, only a 75% recovery was required at the CHT step to achieve 60% overall process recovery. Most commercial IgG processes achieve 50-60% overall process recovery, unless initial aggregate levels are high, in which case overall process recovery may be 25% or less. Data from this stage were evaluated in accordance with the objective of obtaining material suitable for clinical qualification, where the process may not require peak economic efficiency of the process may not be required during process development.

Because CM1 shares an important chemical feature with LM1—weak binding to a cation exchanger—and because LM1 experienced problems with PEG under some conditions, additional experiments were earned out with CM1 to determine if it showed similar sensitivity. CM1 that eluted from CHT in 10% PEG-600 was water-clear upon elution and maintained clarity at room temperature, but rapidly became turbid al 4° C. Turbidity was reversed immediately by dilution with 1 M glycine, as was observed for LM1, with the result that it was determined to be advisable to collect CM1 directly into 1.0 M glycine diluent (1 part sample to 2.3 parts 1.0 M (unbuffered) glycine, pH 7). After dilution, no more solubility issues were observed, but it was deemed prudent to recommend that the next step be commenced within 24 hours after completion of the CHT step. Possible cold and/or insolubility issues with CM1 were considered. Although PEG, as used in the CHT step, does not create novel solubility phenomena; it intensifies phenomena that already exist. Furthermore, it was determined that great care should be exercised with previously frozen material to ensure that it is thoroughly resolubilized before any type of processing.

The specification that the sample be brought to 18-23° C. at all steps of this process may be precautionary. Depending on qualifying experiments performed with material taken directly from storage at 4° C., it may be possible to begin with materials at 4° C., making the entire process faster and more convenient. Temperature solubility curves, from 4° C. to about 23° C., are developed for the anticipated bottling concentration, if known, or for 20 mg/ml if the anticipated bottling concentration is not yet known.

PEG-600 and PEG-1000 can be used interchangeably in this process. The effect of PEG-1000 is slightly stronger than PEG-600 and will cause the antibody to elute a little later, and similarly enhance removal of aggregate. If PEG is omitted entirely, the antibody elutes much earlier, with wash elution/elution setpoints of about 75 mM phosphate and 235 mM phosphate, respectively.

The current glycine level is precautionary and can probably be reduced without risk to the product, but this should be verified experimentally before implementation at large scale.

The hold time after CIIT can probably be extended to a week but should be verified experimentally. One way to evaluate longer holds would be to check turbidity (spectrophotometrically at 600 nm) and analytical size exclusion profiles (for aggregate content), both on a daily basis.

As described above, virus inactivation by the solvent/detergent method can be performed while the antibody is bound to CHT or after elution from CHT.

II. Intermediate Purification by Anion Exchange Chromatography

Intermediate purification by anion exchange chromatography was commenced within 24 hours of completing the initial purification on CHT.

Conditions and Reagents for Anion Exchange Chromatography
Media/column: CIM® QA monolith (8 ml)
Flow rate: up to 10 CV per minute.
Buffer A: 50 mM Tris, 1.0 M glycine, 2 mM EDTA, pH 8.0
Buffer B: 50 mM MES, 10 mM NaCl, 1.0 M glycine, pH 6.2
Buffer C: 50 mM MES, 500 mM NaCl, pH 6.2
Buffer D: 1.0 M NaOH
Buffer E: 0.01 M NaOH or 20% ethanol

Anion Exchange Chromatography

A solution of 1 M Tris, pH 8.0 was added to the pooled fractions collected from CHT, at 5% v:v, to yield a final Tris concentration of 50 mM. The column was equilibrated in Buffer A. Sample solution was loaded on the column by in-line dilution of 1 part sample solution to 2 parts Buffer A. This sample dilution resulted in a total dilution of 10×the volume of sample solution eluted from the CHT step. The capacity of the column was expected to be at least 30 mg IgM per ml of monolith (media), and the alkaline pH was expected to further increase binding capacity. The column was washed with Buffer B (Wash 1), which produced a small peak containing a variety of host cell proteins (HCP). The column was then washed with 71% buffer B, 29% buffer C (Wash 2) which produced a large contaminant peak that may also have contained some IgM. MES buffer, as used in this Example, is zwitterionic and can provide good buffering for both anion and cation exchanger.

Sample was eluted using a 5 CV linear gradient to reach 53% Buffer

B, 47% Buffer C, and the column was then held at 53% Buffer B, 47% Buffer C until the sample peak was fully eluted. Fractions collected beginning at 10% of maximum peak height and continuing until peak descends to 10% of peak height (trailing side), were pooled. It was expected that any remaining aggregate eluted on the trailing side. The column was cleaned with 100% Buffer C, which produced a large peak containing a small amount of IgM mixed with several contaminants, followed by a succession of other small contaminant peaks. Buffer C could be formulated with 1 M NaCl, instead of 500 mM CaCl, for belter cleaning, although any mixtures or gradients would have to be adjusted for the higher NaCl concentration. The column was then sanitized with Buffer D and stored in Buffer E. This intermediate purification step was complete in less than 1 hour. The product (CM1) eluted from the anion exchanger in about 0.5 M glycine and an average concentration of about 225 mM NaCl, which was slightly higher than SAM6 (Example 1, above) or LM1 (Example 3, below). Purity after this step was about 95-98% IgM. Recovery for this step was about 90%.

The 8 ml CIM QA monolith column was oversized for the amount of IgM recovered from the CHT step at the feed volumes recited above. In another experiment, it was found that a 1 ml monolith (a stack of 3×0.34 ml disks) run at 4 ml/min, bound all the IgM from a 5 ml CHT column loaded with 250 ml of cell culture supernatant, which suggested that an 8 ml monolith could retain the IgM obtained from a CHT column loaded with at least 2 liters of cell culture supernatant. As noted above, EDTA in the equilibration buffer was expected to remove any calcium that may have been picked up by the product during the CHT step, and a pH of 8 was expected to increase the binding capacity of the media. Wash and elution steps were carried out at pH 6.2 to enhance removal of HCP, and to provide an eluted sample that would be directly compatible with buffers in the following cation exchange step. However, it was recommended that the next step be performed as soon as possible, preferably within 24 hours, so that the eluted sample remains at pH 6.2 for the shortest time possible. Raising the pH of the solution for use in the following cation exchange step was not practical, as it would have reduced binding efficiency, and the CM1 IgM binds weakly to cation exchange media under even the best of circumstances.

III. Polishing Purification by Cation Exchange Chromatography

Cation exchange chromatography was commenced within 24 hours of the anion exchange step (above).

Conditions and Reagents for Cation Exchange Chromatography
Media/column: CIM SO3 monolith (8 ml)
Flow rate: up to 10 CV per minute.
Buffer A: 10 mM citrate, 1.0 M glycine, pH 6.2
Buffer B: 250 mM citrate, pH 6.2
Buffer C: 1.0 M NaOH
Buffer D: 0.01 M NaOH or 20% ethanol

Cation Exchange Chromatography

Sample solution (pooled fractions from anion exchange) was allowed to reach room temperature (18-23° C.). The column was equilibrated in Buffer A. Sample was loaded by in-line dilution of 1 part sample, 9 parts Buffer A. Note that Buffer A contains 10 mM citrate, which is different from the Buffer A used for cation exchange chromatography in the other Examples. Capacity of the media appeared to be at least 30 mg IgM per ml of monolith. The column was washed with 2-5 CV Buffer A, (Wash 1:2 CV is sufficient, no more than 5 CV). Sample was eluted using a 5 CV linear gradient to reach 12% Buffer B, and the column was then held at 12% Buffer B until the sample peak was fully eluted. IgM eluted clear, in a very sharp peak. Fractions collected beginning at 10% of maximum peak height and continuing until the peak descends to 10% of peak height (trailing side) were pooled. Because it was expected that aggregates eluted in a long low peak on the trailing side, beginning at about 5% of maximum peak height, fractions collected after about 10% of maximum peak height on the trailing side, were not pooled with fractions collected from the main peak. A solution of 500 mM phosphate pH 7 was added to the pooled main peak fractions at 10% v:v, to raise pH and conductivity. The resulting solution of highly purified IgM, contained about 25 mM citrate, 50 mM phosphate, 800 mM glycine, pH ˜6.7, was stored at 4° C.; alternately, fractions can be collected directly into the phosphate diluent (500 mM phosphate pH 7). The column was cleaned with Buffer B, which produced a small peak, principally containing aggregates. The column was sanitized using Buffer C and stored in Buffer D.

This step was completed in less than 1 hour, but could be slowed

down if desired. It was determined that reducing the flow rate will neither improve column performance nor diminish it. Total recovery was about 90%, and purity of the fractions collected from the main elution peak was greater than 99% IgM.

Once started, the final purification step using cation exchange chromatography was complete without interruption, because conditions for cation exchange exposed IgM CM1 to conditions that favor aggregation (low pH and extremely low solution conductivity), and while glycine in the solution can improve solubility, glycine only reduced the risk of aggregation but did not eliminate it.

The present 8 ml column was oversized for the amount of IgM that was recovered at the CHT step with the feed volume described above. In a separate experiment, a 1 ml monolith (a stack of 3×0.34 ml disks) was capable of binding all the IgM eluting from the anion exchange step following a 5 ml CHT column loaded with 250 ml of cell culture supernatant. This result suggested that an 8 ml monolith could retain and release the IgM produced in at least 2 liters of cell culture supernatant loaded on CHT (“CHT feed”).

### Example 3

### Purification Procedure for LM1

I. Capture and Initial Purification by Hydroxyapatite Chromatography

LM1 IgM was purified from a starling material of one (1) liter of

clarified cell culture supernatant, containing approximately 200 μg IgM/ml of cell culture supernatant. First, cell culture supernatant was filtered through a 0.22 micron (0.22 μm) filter. A solution of 500 mM Na phosphate, pH 7.0, was added al 1% v:v, to yield a solution having a final phosphate concentration of 5 mM. If the supernatant already contained phosphate, then the minimum amount of 500 mM Na phosphate, pH 7.0 necessary to yield a phosphate concentration at least 5 mM, was added to the filtered supernatant. The solution pH was measured and, if pH was below pH 6.8, a solution of 1 M Tris, pH 8.0 was added to yield a final pH of 6.8-7.2. The sample solution was allowed to reach room temperature (18-23° C.).

Conditions and Reagents for Hydroxyapatite Chromatography
Media/column: CHT type II, 40 micron, ATOLL 11.3×100 mm column
Flow rate: 100-200 cm/hr (1.67-3.34 ml/min on Atoll column)
Buffer A: 10 mM sodium phosphate, 10% PEG-600, pH 7.0
Buffer B: 500 mM sodium phosphate, 10% PEG-600, pH 7.0
Buffer C: 50 mM Tris, 1.0 M glycine, 2 mM EDTA, pH 8 (+/−0.2)
Buffer D: 600 mM KPO₄, pH 7
Buffer E: 1.0 M NaOH
Buffer F: 0.1 M NaOH, or 20% ethanol, 5 mM sodium phosphate pH 7

Hydroxyapatite Chromatography

The column was equilibrated in Buffer A. Sample solution was applied in 100 column volumes (CV). The column was washed with 2-5 CV Buffer A (Wash 1: 2 CV is sufficient, no more than 5 CV, no need to wash to baseline.) The column was then washed with 20% buffer B (100 mM phosphate, 10% PEG-600), where a large peak eluted in this wash (Wash 2); the peak included host cell proteins (HCP). The column was washed with 20% Buffer B until readings returned to baseline, as “washing to baseline” was important for optimal IgM purification during this step.

Although the contents of the peak eluted during this second wash step sometimes indicated apparent product losses up to 5%, these products were likely to be IgM fragments. However, when product loss seemed excessive, the concentration of Buffer B was reduced, but with the understanding that reducing Buffer B may increase contamination by HCP (i.e. less complete removal of HCP during the wash step, resulting in HCP carryover to other steps). Alternately, if little or no product loss was observed, the phosphate concentration during the wash step was increased, which returned the readings (UV absorbance at 280 nm) back to baseline in a lower, wash volume and also removed more HCP.

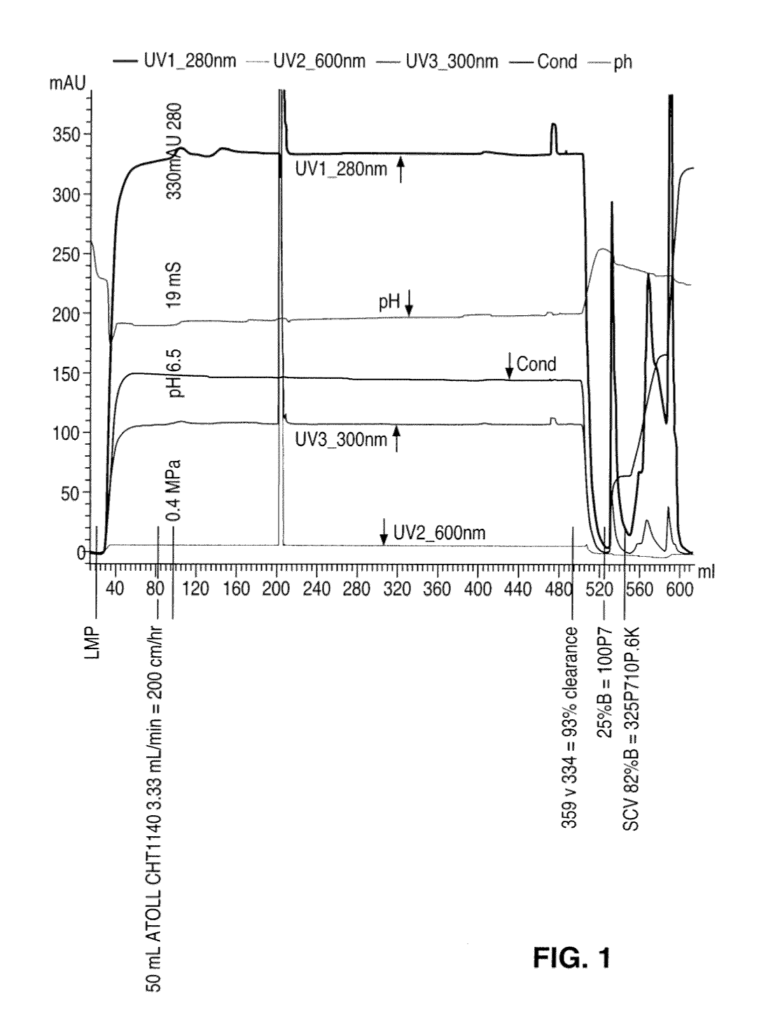

Elution was carried out with a 5 CV linear gradient to reach 65% B (325 mM phosphate, 10% PEG-600), after which point the column was held at 65% B until the product peak eluted. Fractions of 0.5 CV were collected directly into 1.15 CV of 1 M glycine (Buffer C, 50 mM Tris, 1.0 M glycine, 2 mM EDTA, pH 8). The eluting peak was water-clear, and remained so when diluted with glycine. Fractions were collected from about 10% of eluted peak height on the leading side, but only to the point where the shoulder of a contaminant peak begins to appear on the trailing side (see reference profile for LM1 elution from CHT, presented at FIG. 1). Although aggregates may have begun to elute on the trailing side of the peak, this collection/pooling strategy should have excluded aggregates. It was determined that, although the endpoint of the gradient could be reduced slightly, to provide better product purity, perhaps to as low as 300 mM phosphate, it was also understood that because contaminants arc eliminated in later process steps, there was no compelling reason to pursue this strategy at this stage. Fractions were stored at 4° C. The column was cleaned with 5-10 CV Buffer D, sanitized using Buffer E, and stored in Buffer F.

In this example, sample dilution as described in Examples 1 and 2

above, was omitted because dilution doubled the sample loading time and the lower sodium chloride content of the diluted sample allowed more contaminants to bind, yielding a less pure IgM fraction. Results indicated that most of the IgM is bound when 100×volume is applied to 1×volume of CHT (i.e., 100 CV sample solution), although flow-through fractions should be retained during the first few runs, until the relationship between sample load volume and column capacity can be verified. If efficient binding is confirmed, then the loading volume may be increased. If significant product losses are detected in the later flow-through fractions, then reduce the sample application volume accordingly. The use of different cell culture media with a different product concentration will require independent determination of dynamic binding capacity.

Initial purification on CHT required about 6 hours, at a flow rate of 200 cm/hr on a 10 cm bed height, including 5 hours required for sample loading. It was determined that process time will increase in direct proportion with bed height, where doubling bed height will double process time, which indicated that CHT columns for initial purification should probably not exceed 20 cm bed at full process scale, where 15 cm is adequate, and 10 cm may be adequate, depending on the ability to consistently obtain good packing quality. Sample purity after elution from CHT was as high at 90%. Because initial purification on CHT also provided the major aggregate removal step during IgM purification, aggregate concentration after elution from CUT was less than 1%, as verified by size exclusion chromatography (SEC). In cases where aggregate concentration was greater than 1%, the concentration of Buffer B was reduced, e.g. to 60% Buffer B as described above), although results from SEC consistently indicated aggregation concentrations below 1%, such that there was no apparent reason for making process adjustments at this stage.

Comments on Initial Purification

It was determined that PEG-600 and PEG-1000 can be used interchangeably, at the same concentration, in the CHT step. The effect of PEG-1000 is slightly stronger and will cause the antibody to elute a little later, and similarly enhance aggregate removal; however, PEG-600 has a lower melting point and is slightly less viscous. When PEG was omitted entirely, the antibody elutes much earlier, with wash elution/elution setpoints of 50 mM phosphate and 210 mM phosphate, respectively, and little aggregate was removed. For purposes of developing processes for purifying products for clinical use, it will be worthwhile to investigate how much the PEG concentration can be reduced without sacrificing aggregate removal. Alternatively, or in addition, PEG-400 could be substituted, which would simplify buffer preparation since PEG-400 is liquid at room temperature.

As noted previously, virus inactivation by the solvent/detergent method can be performed while the antibody is bound to CHT or after elution from CHT.

II. Intermediate Purification by Anion Exchange Chromatography

Conditions and Reagents for Anion Exchange Chromatography
Media/column: CIM QA monolith (8 ml)
Flow rate: up to 10 CV per minute
Buffer A: 50 mM Tris, 1 M glycine, 2 mM EDTA, pH 8.0
Buffer B: 10 mM sodium phosphate, 1.0 M glycine, pH 7.0
Buffer C: 500 mM sodium phosphate, pH 7
Buffer D: 1.0 M NaOH
Buffer E: 0.01M NaOH or 20% ethanol

Anion Exchange Chromatography

The sample was allowed to reach room temperature (18-23° C.). The column was equilibrated in Buffer A. A flow rate of 2.5 CV/min was routinely used for 8 ml monoliths at 2.5 CV/min, while measurements of sample capacity were run at 12 CV/min. Sample was loaded on the column by in-line dilution of 1 part sample solution to 2 parts Buffer A, representing a total dilution of 10×the volume of sample solution eluted from the CHT step. Glycine was included in the loading solutions to improve antibody solubility and suppress IgM aggregation at full sample dilution. The capacity of the column was expected to be at least 30 mg IgM per ml of monolith. The column was washed with Buffer B (Wash 1) and then washed with up to 5 CV 85% Buffer B, 15% Buffer C (Wash 2) which produced a small peak but did not result in significant loss of IgM.

Sample was eluted using a 5 CV linear gradient to reach 51% Buffer B, 49% Buffer C, after which time the column was held at 51% Buffer B, 49% Buffer C until the sample peak was fully eluted. LM1 eluted at about 250 mM sodium phosphate (in approximately 0.5 M glycine) Fractions containing IgM eluted clear. Fractions collected beginning at 10% of maximum peak height and continuing until the peak descended to 10% of peak height (trailing side) were pooled. Any remaining aggregate were expected to elute on the trailing side. The column was then cleaned with 100% Buffer C, which produced a small sharp peak containing a small amount of IgM, mixed with several contaminants, followed by a succession of other small contaminant peaks. The column was sanitized with Buffer D and stored in Buffer E. This intermediate purification step was completed in less than one hour, although it could be run more slowly if desired.

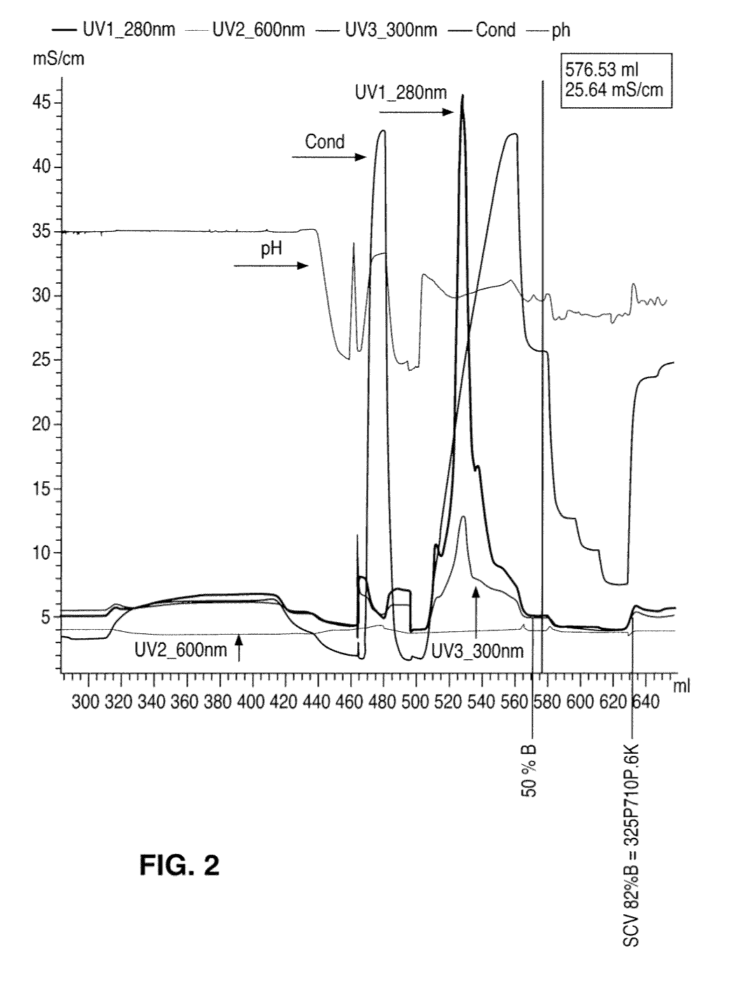

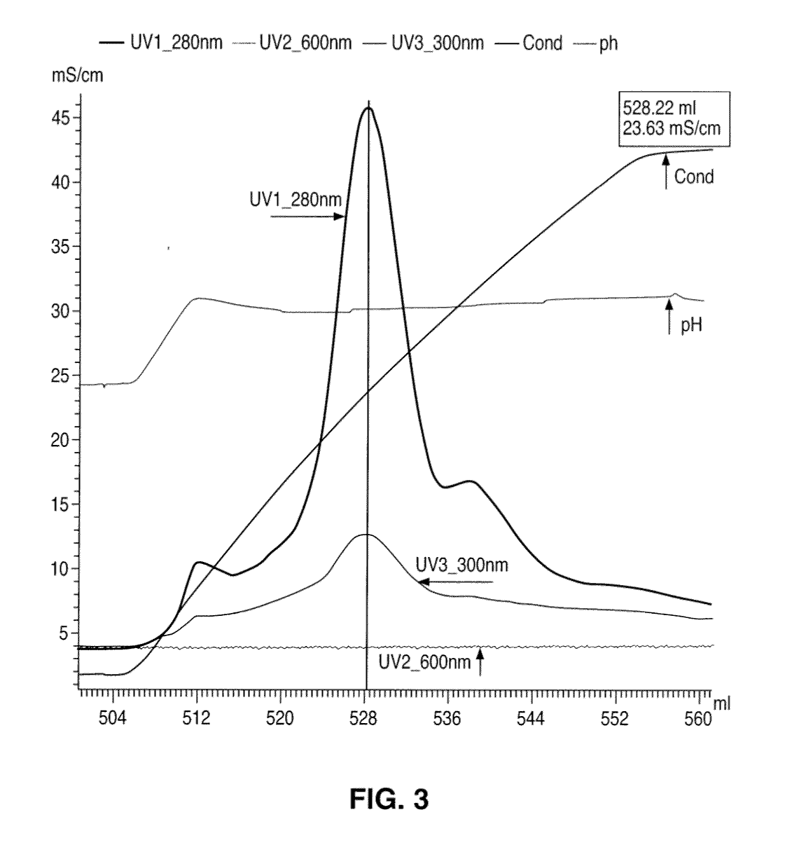

FIGS. 2 and 3 present reference profiles for intermediate purification of LM1 by anion exchange chromatography, under the specific conditions set forth below, where FIG. 2 presents a reference profile for the entire purification step, and FIG. 3 presents a high resolution profile of the elution peak during intermediate purification of LM1 by anion exchange chromatography,

Running conditions for onion exchange chromatography of LM1 in FIGS. 2 and 3
CIM® QA, 3×0.34 ml disks stacked in a single housing, 4 ml/min
Buffer A: 50 mM Tris, 1 M glycine, 2 mM EDTA, pH 8
Buffer B: 10 mM NaPO₄, 1 M glycine, pH 7
Buffer C: 500 mM NaPO₄, pH 7
Equilibrate column
Load sample of pooled fractions from CUT (already diluted to 3.3×with buffer A) by in-line dilution of 1 part sample, 2 parts Buffer A
Wash with Buffer A
Wash with Buffer B
Elute: 48 CV LG to 100% B

As noted above, EDTA in the equilibration buffer was expected to remove any calcium that may have been picked up by the product during the CHT step, as CHT has the capacity to remove non-calcium metals from protein preparations and replace them with calcium. The loading solution was maintained at pH 8 to increase the binding capacity of the media. Wash and elution steps were carried out at pH 7.0 to enhance removal of HCP, and to provide an eluted sample that would be directly compatible with buffers in the following cation exchange step. If a viral filtration step has not been carried out and is desired, viral filtration can take place after anion exchange.

III. Polishing Purification of LM1 by Cation Exchange Chromatography

Conditions and Reagents for Cation Exchange Chromatography
Media/column: CIM SO3 monolith (8 ml)
Flow rate: up to 10 CV per minute; lower flow rates caused no loss of performance, nor significant gain.
Buffer A: 10 mM sodium phosphate, 1 M glycine, pH 7
Buffer B: 500 mM sodium phosphate, pH 7
Buffer C: 1.0 M NaOH
Buffer D: 0.01 M NaOH or 20% ethanol

Cation Exchange Chromatography

Sample solution (pooled fractions from anion exchange chromatography) was allowed to reach room temperature. The column was equilibrated with Buffer A. Sample was loaded by in-line dilution of 1 part sample solution, 9 parts Buffer A. The capacity of the column under these conditions appeared to be at least 30 mg IgM per ml of monolith. The column was washed in 2-5 CV Buffer A (Wash 1: 2 CV is sufficient, no more than 5 CV). Sample was eluted using a 5 CV linear gradient to reach 15% Buffer B (75 mM phosphate), after which lime the column was held at 15% Buffer B until peak was fully eluted. The fraction containing IgM eluted clear. It was determined that, since LM1 eluted at a low conductivity value (low salt concentration) NaCl could be added, e.g., to a final concentration of 0.1 M, to stabilize the antibody and prevent aggregation, where NaCl would be added immediately after elution or by collecting fractions directly into a high-salt diluent. Collected fractions were stored at 4° C. The column was cleaned using Buffer B, which produced a peak containing a significant amount of IgM. The column was sanitized using Buffer D, and stored in Buffer E.

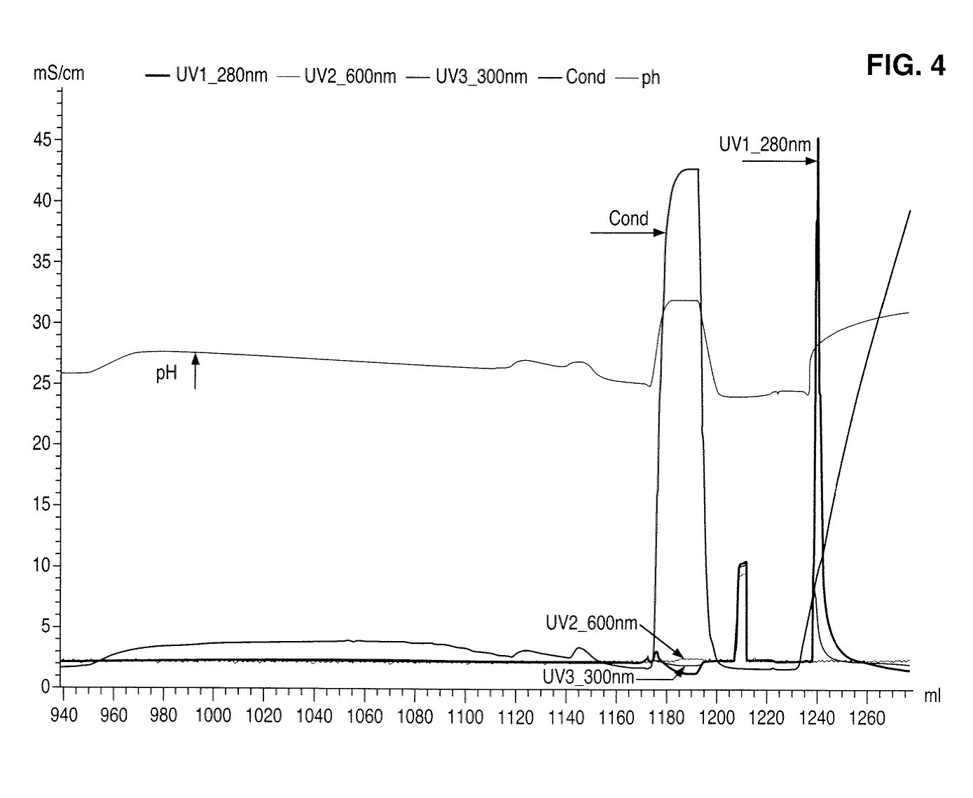

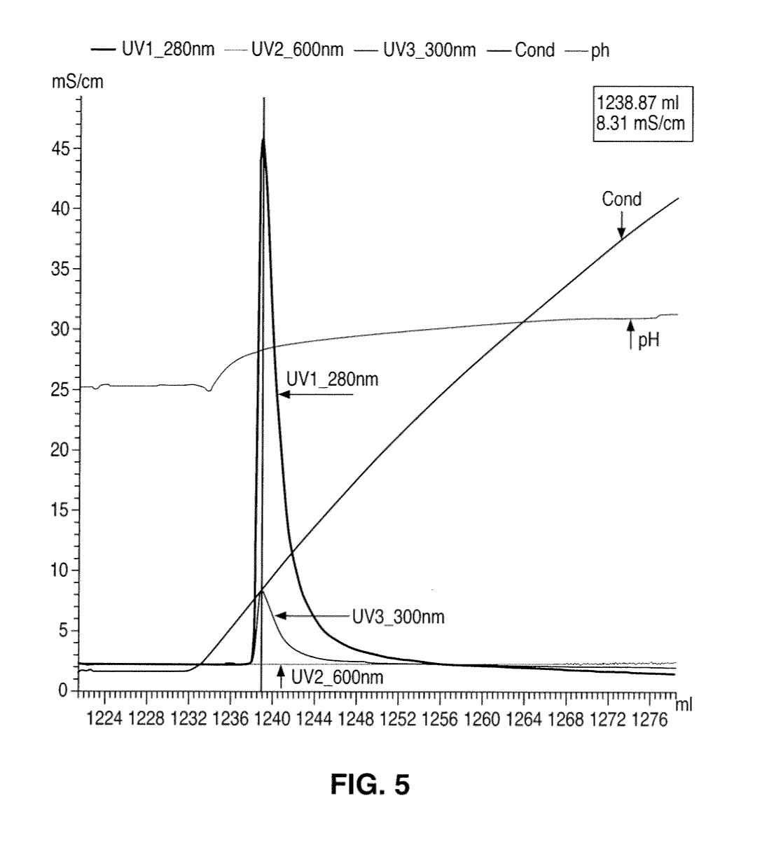

FIGS. 4 and 5 present reference profiles for polishing purification of LM1 by cation exchange chromatography, under the specific conditions set forth below, where FIG. 4 presents a reference profile for the entire purification step, and FIG. 5 presents a high resolution profile of the elution peak during polishing purification of LM1

Running Conditions for Polishing Purification in FIGS. 4 and 5
CIM® SO3, 3×0.34 ml disks stacked in a single housing, 4 ml/min
Buffer A: 10 mM NaPO₄, 1 M glycine, pH 7
Buffer B: 500 mM NaPO₄, pH 7
Equilibrate column
Load sample of pooled fractions from anion exchange by in-line dilution of 1 part sample, 9 parts Buffer A
Wash with Buffer A
Elute: 48 CV LG to 100% B

For LM1 purification by cation exchange chromatography, the

shape of the elution peak was atypical in its lack of definition on the trailing side (See FIGS. 4 and 5, especially FIG. 5). Therefore, in order to avoid collected aggregates, pooling specifications were set to exclude most of the trailing portion, e.g., fractions were collected only until the peak had decreased to 25% of maximum peak height on the trailing side, in order to ensure that no aggregates that may have been eluting on the trailing side were collected with the IgM peak. The effectiveness of (his strategy was evaluated by high performance size exclusion chromatography (HPSEC) analysis of the LM1 peak fractions after polishing purification as disclosed in Example 4, which could not detect the presence of aggregates (i.e., aggregate levels were below the limits of detection, which is about 0.1%), confirming that this approach resulted in a preparation free from IgM detectable aggregates.

### Example 4

### Analytical Size Exclusion Chromatography of Purified LM1

Analytical size exclusion chromatography (SEC) of IgM-containing

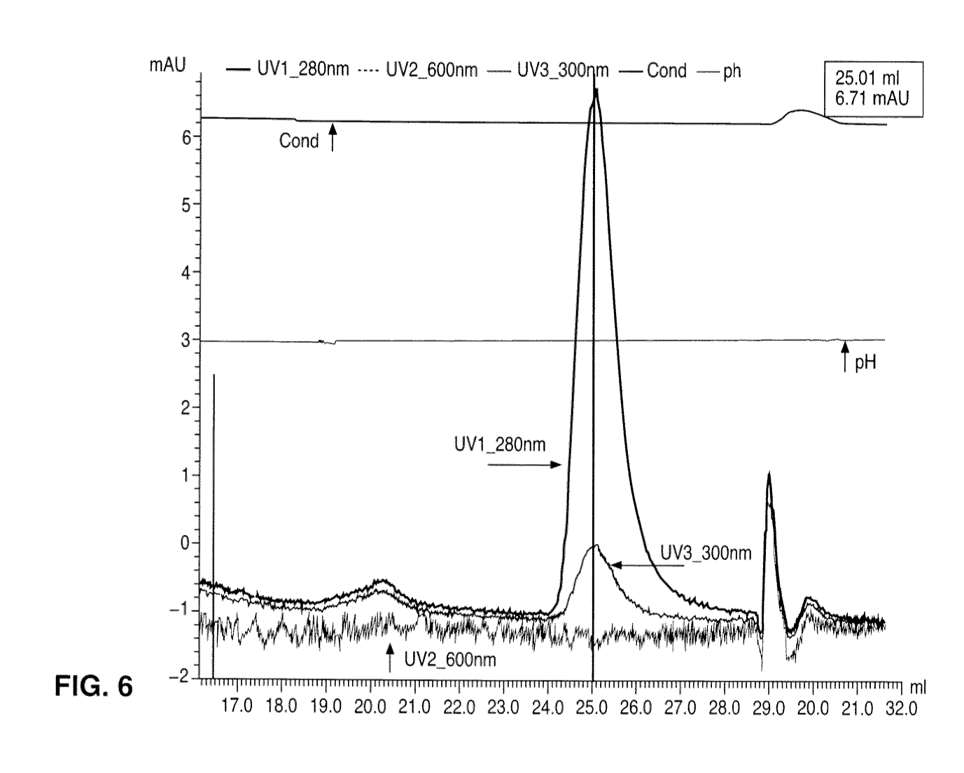

fractions from polishing purification chromatography was carried out to evaluate the size (molecular radius) of the purified IgM, as well as the purity and other features of the IgM-containing samples. A sample of 100 μl LM1 from the pooled fractions collected from the elution peak during polishing purification of LM1 by cation exchange chromatography as described in Example 4. above, was loaded on GSW4000 SEC media (Toso BioSep, Stuttgart, Del.), run with SEC Buffer (25 mM MES, 0.5 M glycine, 0.5M NaCl, 0.2 M arginine, pH 6.8) at a flow rate of 0.5 ml/mind. SEC eluates were analyzed by measuring absorbance at 280 nm and 300 nm to measure total protein and at 600 nm to measure turbidity. Conductivity of the solutions was also measured. The analytic SEC profile for this sample is presented at FIG. 6. LM1 eluted in a single peak, indicating an almost complete lack of contaminants such as IgM fragments and IgM aggregates. The center of the LM1 peak elutes at 8.55 minutes after injection (FIG. 6). The elution time of LM1 was 1.03 minutes later than the SEC elution peak observed for purified CM1 (data not shown) and about 0.85 min later than the SEC elution peak for purified SAM6 (data not shown). Given that the SEC buffer was formulated specifically to prevent nonspecific hydrogen bonding, as well as ionic and hydrophobic interactions, these results indicated that LM1 IgM has a smaller hydrodynamic radius than the other two antibodies (CM1 and SAM6). LM1 also shows an unusual elution profile from cation exchange media; the cation exchange elution profile, and sensitivity to pH observed for LM1 were noted.

During SEC of the LM1-containing fraction, an elution peak seen at 20 minutes after injection was a buffer artifact, as demonstrated by the parallel traces for measurements of A₂₈₀ and A₃₀₀. Artifactual peaks observed at 29 and 30 minutes after injection were caused by changes in the refractive index as sample buffer eluted from the column, as indicated by the parallel traces for measurements of A₂₈₀, A₃₀₀, A₆₀₀ and a simultaneous increase in the conductivity measurement.

### Example 5

### Purification Procedure for SAM6

I. Sample Capture and Initial Purification by Hydroxyapatite Chromatography.

The procedure of SAM6 purification was performed by substituting 2M urea for 1M glycine in the process of Example 1. Urea-containing buffers were filtered through an anion exchange filter, such as Sartobind Q (SingleStep nano 1 ml), before use. Use of ACS grade urea, or better, is highly recommended. Urea-containing buffers were be assigned an expiration of no greater than 7 days as a precaution to minimize the probability of carbamylation.

SAM6 IgM was purified from a starting material of one liter of

clarified cell culture supernatant, containing approximately 200 μg 1 gM/ml of cell culture supernatant. First, cell culture supernatant was filtered using a 0.22 micron (0.22 μm) filter, and followed by addition of 500 mM Na phosphate, pH 7.0, at 1% v:v, to yield a final phosphate concentration of 5 mM. If the sample already contained phosphate, then the minimum amount of 500 mM Na phosphate, pH 7, necessary to yield a phosphate concentration of at least 5 mM was added to the filtered supernatant. A solution of 1 M Tris, pH 8.0, was added at 1% v:v, to yield a final concentration of 10 mM Tris, which was expected to yield a final pH of 6.8 to 7.2. The sample solution was allowed to reach room temperature (18-23° C.).

Conditions and Reagents for Hydroxyapatite Chromatography
Media/column: CHT type II, 40 micron, ATOLL 11.3×100 mm column
Flow rate: 100-200 cm/hr (1.67-3.34 ml/min on Atoll column)
Buffer A: 10 mM sodium phosphate, 10% PEG-600, pH 7.0
Buffer B: 500 mM sodium phosphate, 10% PEG-600, pH 7.0
Buffer C: 10 mM sodium phosphate, 2 M urea, 2 mM EDTA, pH7
Buffer D: 600 mM KPO4, pH 7
Buffer E: 1.0 M NaOH
Buffer F: 0.1 M NaOH, or 20% ethanol, 5 mM sodium phosphate pH 7

Hydroxyapatite Chromatography.

The column (ceramic hydroxyapatite CHT™ type II 40 micron

(Bio-Rad Laboratories, Hercules, Calif.), 11.3×100 mm column pre-packed by ATOLL Gmbh) was equilibrated in Buffer A (above). The sample was applied in 100 column volumes (100 CV) of Buffer A at approximately 0.1 ml/min. After (he sample was loaded, the column was washed with between 2 to 5 CV Buffer A (Wash 1). The column was then washed with 25% buffer B (125 mM phosphate, 10% PEG-600) until readings returned to baseline values as determined by measuring absorption at 280 nm, A₂₈₀ (Wash 2).

Flow was stopped and a Sartobind Q membrane anion exchange

filter was connected at the bottom of the CHT column and then flow was resumed under wash conditions until the monitor indicates that the Q cartridge reached equilibrium. (A 1 ml cartridge accommodates a 10 mL CHT column, likely 10 times that, possibly much more.).

Sample was eluted from the column with a one (1) CV linear

gradient to 70% Buffer B (350 mM phosphate, 10% PEG-600), and the column was then held at 70% Buffer B until the product peak eluted. Fractions of 0.5 CV or less were collected and the pool was diluted to 3.3 limes the original pool volume with Buffer C. The eluting peak was water-clear, and remained so when diluted with urea. Fractions were stored at 4° C. As recommended, fractions from 10% of maximum peak height on the leading side, to 10% maximum peak height on the trailing side were pooled for further purification. It was expected that this collecting/pooling strategy excluded aggregates that may begin to elute on trailing side of the sample peak. The CHT column was cleaned with 5-10 CV Buffer D, sanitized with Buffer E, and stored in Buffer F.

II. Intermediate Purification by Anion Exchange Chromatography.

Intermediate purification by anion exchange chromatography was

commenced within 24 hours of completing the initial purification on CHT and performed at a minimum flow rate of 4 ml/min on an AKTA Explorer 100.

Conditions and Reagents for Anion Exchange Chromatography
Media/column: CIM® QA monolith (8 ml)
Flow rate: up to 10 CV per minute.
Buffer A: 10 mM sodium phosphate, 2 M urea, pH 7
Buffer B: 50 mM MES, 10 mM NaCl, 2 M urea, pH 6.2
Buffer C: 50 mM MES, 500 mM NaCl, pH 6.2
Buffer D: 1.0 M NaOH
Buffer E: 0.01 M NaOH or 20% ethanol

Anion Exchange Chromatography

A solution of 1 M Tris, pH 8.0 was added to the pooled fractions

collected from CHT, at 5% v:v, to yield a final Tris concentration of 50 mM, and the sample solution was allowed to reach room temperature (18-23° C.).

The column containing eight (8) ml strong anion exchanger CIM® QA monolith was equilibrated in Buffer A, and the sample solution was loaded on the column by in-line dilution as follows: 1 part sample (supplied by Pump A) to 4 parts Butter A (supplied by Pump B). This sample dilution resulted in a total dilution of 10×the volume eluted from the CHT column. The expected column capacity of the QA monolith used for this step was about 30 mg IgM per ml of monolith. The column was washed with Buffer B (Wash 1) and then washed with 95% Buffer B, 5% Buffer C (wash 2).

Sample was then eluted using a 5 CV linear gradient to reach 52% Buffer B, 48% Buffer C, and the column was held at 52% Buffer B, 48% Buffer C until the sample peak was fully eluted. Fractions collected beginning at 10% of maximum peak height on the leading edge until 10% of maximum peak height on the trailing edge, were pooled. It was expected that any remaining aggregate eluted on the trailing side. The column was cleaned with 100% Buffer C, which produced a small sharp peak containing a small account of IgM mixed with several contaminants, followed by a succession of other small contaminant peaks. The column was sanitized with Buffer D and stored in Buffer E. This intermediate purification step was completed in less than one hour.

Comments on Intermediate Purification

Note that 1-2 M NaCl will possibly support better column cleaning. The only disadvantage is the preparation of this additional buffer. Occasional cleaning of the column with benzonase to remove accumulated DNA may extend column life (for example, every 10 runs, or whenever backpressure becomes excessive).

Purification should proceed to the next step as soon as reasonably possible to limit product exposure to urea. The alkaline pH of the Tris urea buffer increases the risk of carbamylation but this is offset by the brief duration of contact. An early version of the process used pH 7 phosphate and the capacity specs were set at this pH, so the Tris pH 8 can probably be substituted for the original buffer without loss of purification performance.

III. Polishing Purification by Cation Exchange Chromatography.

Cation exchange chromatography was commenced within 24 hours of the anion exchange step (above) and performed at a minimum flow rate of 4 ml/min on an AKTA Explorer 100.

Conditions and Reagents for Cation Exchange Chromatography
Media/column: CIM® SO3 monolith (8 ml)
Flow rate: up to 10 CV per minute.
Buffer A: 10 mM sodium phosphate, 2 M urea, pH 7
Buffer B: 20 mM citrate, 2 M urea, pH 6.2
Buffer C: 250 mM citrate, pH 6.2
Buffer D: 1.0 M NaOH
Buffer E: 0.01 M NaOH or 20% ethanol

Cation Exchange Chromatography

Once started, the Final purification step using cation exchange chromatography was completed without interruption.

Sample (pooled fractions from anion exchange) was allowed to reach room temperature (18-23° C.). The column was equilibrated in Buffer A. Sample was loaded by in-line dilution of 1 part sample solution to 9 parts Buffer A. Capacity of the CIM SO3 media appeared to be about 30 mg IgM/ml. The column was washed with 2-5 CV Buffer A (Wash 1: 2 CV is sufficient, no more than 5 CV), and then washed with 5 CV 95% Buffer B, 5% Buffer C (Wash 2) which produced a small peak.

Sample was eluted using a 5 CV linear gradient to reach 60% Buffer B, 40% Buffer C, and the column was then held at 60% Buffer B, 40% Buffer C the sample peak was fully eluted. Fractions collected beginning al 10% of maximum peak height on the leading edge until 10% of maximum peak height on the trailing edge, were pooled. IgM eluted clear. It was expected that any remaining aggregate eluted on the trailing side. A solution of 500 mM phosphate pH 7 was added to the pooled fractions at 10% v:v, to raise the pH, and the solution was stored at 4°. The column was cleaned with Buffer B, which produced a small peak. The column was sanitized in Buffer D and stored in Buffer E.

Comments on Polishing Purification

The IgM should be diafiltered into final formulation soon after purification to remove urea.

### Example 6

### Purification Procedure for LM1

The procedure of LM1 purification was performed by substituting LM1 for SAM6 in the process of Example 5 with certain changes in the buffers used. During the intermediate purification using anion exchange chromatography step. Buffers A and B were prepared as follows:
Buffer A: 50 mM Tris, 2 M urea, 2 mM EDTA, pH 8.0
Buffer B: 10 mM sodium phosphate, 2 M urea, pH 7.0

During the polishing purification by cation exchange chromatography step the second wash was omitted and Buffer B and mixtures of Buffer B and C were replaced with the following Buffer B: 500 mM sodium phosphate, pH 7. The elution step was performed using a 5 CV linear gradient to 15% Buffer 13 (75 ml phosphate). Pooling was conducted from 10% of max peak height to 25% pf max peak height after the peak. NaCl was added to a concentration of 0.1M to stabilize the antibody and discourage aggregation.

1. A process for purification of a protein product from a sample comprising the protein product and aggregates of the protein product, the process comprising:
(a) a first chromatography step comprising the use of a nonionic polymer for removal of the aggregates of the protein product, wherein the nonionic polymer is present at concentrations sufficient to enhance separation of the protein product from the aggregates of the protein product under the chromatography conditions, such that a fraction comprising the protein product substantially free of aggregates is collected after the step;
(b) a step of combining a solubility enhancing additive and the fraction comprising the protein product obtained in the first chromatography step or a subsequently obtained fraction comprising the protein product which fraction is derived from the fraction comprising the protein product obtained in the first chromatography step, wherein the solubility enhancing additive is selected from the group consisting of a zwitterion, a urea compound, and an alkylene glycol; and
(c) a second chromatography step comprising the use of ion exchange chromatography wherein the solubility enhancing additive is present in sufficient concentration to enhance solubility of the protein product and substantially avoid occlusion under the chromatography conditions, and wherein the solubility enhancing additive does not interfere with the second chromatography step, and
wherein the process yields a purified protein product substantially lice of aggregates.

2. The process of claim 1, wherein the sample is a cell culture supernatant.

3. The process of claim 1 wherein the protein product is an immunoglobulin or fragment thereof.

4. The process of claim 3, wherein the immunoglobulin is IgM.

5. The process of claim 1, wherein the solubility enhancing additive is selected from the group consisting of glycine, betaine, urea, ethylene glycol and polyethylene glycol.

6. The process of claim 1, wherein the nonionic polymer of the first chromatography step is polyethylene glycol (PEG).

7. The process of claim 1, wherein the first chromatography step comprises hydroxyapatite chromatography wherein the nonionic polymer is present at concentrations sufficient to enhance separation of the protein product from the aggregates under hydroxyapatite chromatography conditions.

8. The process of claim 7 wherein the nonionic polymer is polyethylene glycol and the solubility enhancing additive is selected from the groups consisting of glycine and urea.

9. The process of claim 8, wherein the fraction collected after the hydroxyapatite chromatography is collected into a composition comprising the solubility enhancing additive.

10. The process of claim 1 wherein the fraction comprising the protein product collected after the first chromatography step is subjected to further separation or purification steps to yield a fraction comprising the protein product derived from the fraction obtained from the first chromatography prior to the step of combining such fraction with the solubility enhancing additive.

11. The process of claim 1, wherein the second chromatography step comprises anion exchange chromatography.

12. The process of claim 1, wherein the second chromatography step comprises cation exchange chromatography.

13. The process of claim 11, wherein the second chromatography step additionally comprises cation exchange chromatography.

14. The process of claim 1, wherein the fraction comprising the protein product obtained from the first chromatography step is combined with a composition comprising the solubility enhancing additive.

15. The process of claim 14, wherein the solubility enhancing additive is a zwitterion.

16. The process of claim 1 comprising a third chromatography step comprising ion exchange chromatography of the fraction obtained from the second chromatography step.

17. The process of claim 16, wherein the second chromatography step is anion exchange chromatography and the third chromatography step is cation exchange chromatography.

18. The process of claim 16, wherein the second chromatography step is cation exchange chromatography and the third chromatography step is anion exchange chromatography.

19. The process of claim 1, wherein the solubility enhancing agent is glycine.

20. The process of claim 4, wherein the first chromatography step comprises hydroxyapatite chromatography wherein the nonionic polymer is present at concentrations sufficient to enhance separation of the IgM from the IgM aggregates under hydroxyapatite chromatography conditions.

21. The process of claim 20 wherein the nonionic polymer is polyethylene glycol.

22. The process of claim 21, wherein the nonionic polymer is polyethylene glycol at a concentration of about 10%.

23. The process of claim 21, wherein the nonionic polymer is polyethylene glycol and the solubility enhancing additive is selected from the group consisting of zwitterion and urea.

24. The process of claim 23, wherein the solubility enhancing additive is glycine.

25. The process of claim 24, wherein the second chromatography step is carried out in the presence of glycine at concentrations of between about 0.5 M and about 1 M, wherein the process yields IgM substantially free of IgM aggregates, wherein the IgM has a purity in excess of about 99%.

26. The process of claim 25, further wherein the fraction collected after the hydroxyapatite chromatography of the first chromatography step is collected into a composition comprising glycine at a concentration of about 1M.
